(19) United States
(12) Patent Application Publication
(10) Pub. No.: US 2024/0360494 A1
(43) Pub. Date: Oct. 31, 2024

(54) METHODS OF CAPTURING TARGET ANALYTES

(71) Applicant: 10x Genomics, Inc., Pleasanton, CA (US)

(72) Inventors: Justin Costa, Union City, CA (US); Felice Alessio Bava, Rome (IT)

(21) Appl. No.: 18/763,579

(22) Filed: Jul. 3, 2024

Related U.S. Application Data
(63) Continuation of application No. PCT/US2023/086185, filed on Dec. 28, 2023.
(60) Provisional application No. 63/436,376, filed on Dec. 30, 2022.

(51) Int. Cl. C12Q 1/6837; C12Q 1/6876

(52) U.S. Cl. CPC C12Q 1/6837; C12Q 1/6876

(57) ABSTRACT

Provided herein are methods, compositions, and kits for capturing a ligation product, in which the method can include covalently reacting a capture moiety of a capture probe with a reactive moiety of the ligation product to form a captured ligation product.

### CROSS-REFERENCE TO RELATED APPLICATIONS

Pursuant to 35 U.S.C. § 119 (e), this application is a continuation of International Application PCT/US2023/086185, with an international filing date of Dec. 28, 2023, which claims the benefit of U.S. Provisional Patent No. 63/436,376, filed on Dec. 30, 2022. The contents of which are incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that provide data for a handful of analytes in the context of an intact tissue or a portion of a tissue, or provides substantial analyte data for dissociated tissue (i.e., single cells), but fails to provide information regarding the position of a single cell in a parent biological sample (e.g., tissue sample).

In general, analytes in a biological sample migrate vertically (e.g., via gravity) from their point of origin to a substrate, for example a substrate that includes a plurality of capture probes with capture domains for capturing the analytes. However, in practice some of the analytes can migrate in other directions (e.g., non-vertical directions such as lateral diffusion), which when captured on an array can lead to a loss of sensitivity when the analyte is not captured proximal to the location from which it originated. Additionally, an analyte may not be captured at all (e.g., the analyte diffuses away from the array). This effect is known as target mislocalization or as used herein “TML”. When TML occurs, sensitivity decreases leading to potential inaccurate localization data.

The present disclosure provides solutions for mitigating TML from a tissue or cell, thereby resulting in higher resolution analyte expression data such as from a single cell and/or a tissue, a tissue section, etc. from a biological sample.

### SUMMARY

Spatial transcriptomics (ST) can be used to reduce a three-dimensional (3D) distribution of molecules within a sample into a two-dimensional (2D) image. To do so, a 2D image can be obtained by capturing target molecules that migrate vertically from their original location (e.g., such as within the sample) onto a capturing slide or array. For high-resolution ST, directional control of this migration would be beneficial. Migration of molecules in non-vertical directions could reduce resolution, accuracy, and/or sensitivity. In one instance, non-vertical migration could result in failure to capture target molecules, which may then be washed away during latter processing steps. In another instance, target molecules may be captured in an incorrect location, as compared to the original location of the target molecule (which can be referred to as molecule mislocalization). Described herein are methods, compositions, and kits that can reduce loss of target molecules and minimize molecular mislocalization. In some embodiments, such methods can provide improved binding or capture of a target molecule, thereby maintaining or enhancing assay sensitivity.

Thus provided herein are methods for capturing a ligation product, the method including: (a) providing a biological sample on an array including a plurality of capture probes, where a capture probe of the plurality of capture probes includes: (i) a spatial barcode and (ii) a capture moiety; and (b) covalently reacting the capture moiety of the capture probe with a reactive moiety of the ligation product to form a captured ligation product, where the ligation product includes a sequence that is substantially complementary to a sequence of an analyte in the biological sample, and where the capture moiety and the reactive moiety react together to form a junction configured for replication by a polymerase.

In some embodiments, the ligation product is generated by ligating a first probe and a second probe, and where the first probe and the second probe each include one or more sequences that are substantially complementary to sequences of the analyte. In some embodiments, the one or more sequences are adjacent to one another. In some embodiments, there is a gap between the one or more sequences, and where the first probe or the second probe is extended until the probes abut prior to ligating.

In some embodiments, the method includes before (b): hybridizing the first probe and the second probe to the analyte; generating the ligation product by ligating the first probe and the second probe; and releasing the ligation product from the analyte.

In some embodiments, the junction includes a click signature linkage, a triazole linkage, an isoxazoline linkage, an S-phosphorothioester linkage, a phosphorothioate linkage, an amide linkage, a disulfide linkage, a boranophosphate linkage, a phosphoramidate linkage, a urea linkage, or a squaramide linkage.

In some embodiments, the method includes before (a): converting a terminal nucleotide of the capture probe to provide the capture moiety. In some embodiments, the converting includes treating the terminal nucleotide with an oxidizing agent to provide the capture moiety including one or more aldehyde moieties. In some embodiments, the terminal nucleotide includes a ribose, a ribonucleotide, a reversibly terminated nucleotide, or a reversibly terminated ribonucleotide.

Also provided herein are methods for capturing a ligation product, the method including: (a) providing a biological sample on an array including a plurality of capture probes, where a capture probe of the plurality of capture probes includes: (i) a spatial barcode and (ii) a capture moiety, or where a first capture probe of the plurality of capture probes includes (i) a spatial barcode and where a second capture probe of the plurality of capture probes includes (ii) a capture moiety; and (b) covalently reacting the capture moiety with a reactive moiety of a ligation product to form a captured ligation product, where the ligation product includes a sequence that is substantially complementary to a sequence of an analyte in the biological sample, and where the capture moiety and the reactive moiety react together to form a junction that is not configured for replication by a polymerase.

In some embodiments, the junction includes a click signature linkage, streptavidin/biotin linkage, an avidin/biotin linkage, a maltose/maltose-binding protein linkage, a carbohydrate/carbohydrate-binding protein linkage, or an antigen/antibody linkage.

In some embodiments, the capture probe includes a cleavable moiety in proximity to the capture moiety, and where the cleavable moiety is configured to be cleaved after forming the junction.

In some embodiments, the capture probe includes a priming region located in proximity to the junction.

In some embodiments, the method includes after (c) generating an extended ligation product by binding to the priming region and extending the capture probe, where the extended ligation product includes a sequence complementary to the sequence of the ligation product; and (d) cleaving the cleavable moiety, thereby releasing the captured ligation product and providing a product lacking the junction.

In some embodiments, the second capture probe includes a cleavable moiety in proximity to a surface of the array.

In some embodiments, the first capture probe includes a priming region located in proximity to the junction.

In some embodiments, the method includes after (c) generating an extended ligation product by binding to the priming region and extending the first capture probe, where the extended ligation product includes one or more sequences that are substantially complementary to sequences of the analyte; and (d) cleaving the cleavable moiety of the second probe, thereby releasing a portion of the captured ligation product from the surface of the array and providing a product, where the product includes one or more sequences that are substantially complementary to sequences of the analyte.

In some embodiments, the method includes determining (i) all or a part of the sequence of the captured ligation product or the product, or a complement thereof, and (ii) the sequence of the spatial barcode, or a complement thereof, and using the determined sequence of (i) and (ii) to identify a location of the ligation product (e.g., a proxy of the analyte) in the biological sample.

Also provided herein are methods for capturing a ligation product, the method including: (a) providing a biological sample on an array including a plurality of capture probes, where a capture probe of the plurality of capture probes includes (i) a spatial barcode and (ii) a capture moiety, or where a first capture probe of the plurality of capture probes includes (i) a spatial barcode and where a second capture probe of the plurality of capture probes includes (ii) a capture moiety; (b) hybridizing a first probe and a second probe to an analyte in the biological sample, where the first probe and the second probe each include one or more sequences that are substantially complementary to sequences of the analyte, and where the second probe includes a reactive moiety; (c) generating a ligation product by ligating the first probe and the second probe, where the ligation product includes the reactive moiety; (d) releasing the ligation product from the analyte; and (e) covalently reacting the capture moiety of the capture probe with the reactive moiety of the ligation product to form a captured ligation product.

In some embodiments, the method includes (f) determining (i) all or a part of the sequence of the captured ligation product, or a complement thereof, and (ii) the sequence of the spatial barcode, or a complement thereof, and using the determined sequence of (i) and (ii) to identify a location of the ligation product (e.g., a proxy of the analyte) in the biological sample.

Also provided herein methods for identifying a location of an analyte in a biological sample, the method including: (a) contacting the biological sample with a first substrate; (b) hybridizing a first probe and a second probe to the analyte, where the second probe includes a reactive moiety that covalently reacts with a capture moiety for a capture probe of a plurality of capture probes, where the capture probe is affixed to the first substrate or where the capture probe is affixed to a second substrate, and where at least one capture probe of the plurality of capture probes includes a spatial barcode; (c) ligating the first probe and the second probe, thereby creating a ligation product, where the ligation product includes a sequence that is substantially complementary to the analyte, and where the ligation product includes the reactive moiety; (d) releasing the ligation product from the analyte; (e) covalently reacting the capture moiety with the reactive moiety of the ligation product to form a captured ligation product; and (f) determining (i) all or part of the sequence of the ligation product, or a complement thereof, and (ii) the sequence of the spatial barcode, or a complement thereof, and using the determined sequences of (i) and (ii) to identify a location of the analyte in the biological sample.

In some embodiments, the capture probe of the plurality of capture probes includes the spatial barcode and the capture moiety. In some embodiments, the capture probe of the plurality of capture probes includes the capture moiety, and where a capture probe of the plurality of capture probes includes the spatial barcode.

Also provided herein are methods of enhancing binding of a ligation product to a capture moiety, the method including: (a) contacting a biological sample with a first substrate; (b) hybridizing a first probe and a second probe to an analyte of the biologicals sample, where the second probe includes a reactive moiety that covalently reacts with a capture moiety of a capture probe of a plurality of capture probes, where the capture probe is affixed to the first substrate or where the capture probe is affixed to a second substrate, and where at least one capture probe of the plurality of capture probes includes a spatial barcode; c) ligating the first probe and the second probe, thereby creating a ligation product, where the ligation product includes a sequence that is substantially complementary to the analyte, and where the ligation product includes the reactive moiety; (d) releasing the ligation product from the analyte; and (c) covalently reacting the capture moiety of the capture probe with the reactive moiety of the ligation product to form a captured ligation product.

In some embodiments, the capture probe is affixed to the first substrate, and where the first substrate includes a plurality of capture probes. In some embodiments, the capture probe is affixed to the second substrate, and where the second substrate includes a plurality of capture probes.

In some embodiments, the method includes aligning the first substrate with the second substrate such that at least a portion of the biological sample is aligned with at least a portion of the plurality of capture probes.

Also provided herein are methods for processing a nucleic acid analyte in a biological sample mounted on a first substrate, the method including: (a) hybridizing a first probe and a second probe to the nucleic acid analyte, where the first probe and the second probe each include a sequence that is substantially complementary to nucleic acid sequences of the nucleic acid analyte, and where the second probe includes a reactive moiety; (b) ligating the first probe and the second probe, thereby generating a ligation product, where the ligation product includes the reactive moiety; (c) aligning the first substrate with a second substrate including an array, such that at least a portion of the biological sample is aligned with at least a portion of the array, where the array includes a plurality of capture probes, where a capture probe of the plurality of capture probes includes a spatial barcode and a capture moiety, or where a first capture probe of the plurality of capture probes includes (i) a spatial barcode and where a second capture probe of the plurality of capture probes includes (ii) a capture moiety; (d) releasing the ligation product from the nucleic acid analyte when at least a portion of the biological sample is aligned with at least a portion of the array; and (c) hybridizing the ligation product to the capture moiety of the array.

In some embodiments, the releasing includes migrating the ligation product from the biological sample to the array. In some embodiments, the migrating includes migrating in a vertical direction between the biological sample and the array.

In some embodiments, the aligning includes: mounting the first substrate on a first member of a support device, the first member configured to retain the first substrate; mounting the second substrate on a second member of the support device; applying a reagent medium to the first substrate and/or the second substrate; and operating an alignment mechanism of the support device to move the first member and/or the second member such that at least a portion of the biological sample is aligned with at least a portion of the array, and such that the portion of the biological sample and the portion of the array contact the reagent medium.

In some embodiments, the alignment mechanism is coupled to the first member, the second member, or both the first member and the second member.

In some embodiments, the alignment mechanism includes a linear actuator, and optionally where the linear actuator is configured to move the second member along an axis orthogonal to a plane of the first member and/or the second member, and/or the linear actuator is configured to move the first member along an axis orthogonal to the plane of the first member and/or the second member, and/or the linear actuator is configured to move the first member, the second member, or both the first member and the second member at a velocity of at least 0.1 mm/sec, and/or the linear actuator is configured to move the first member, the second member, or both the first member and the second member with an amount of force of at least 0.1 lbs.

In some embodiments, at least one of the first substrate and the second substrate include a spacer disposed on the first substrate or the second substrate, where when at least the portion of the biological sample is aligned with at least a portion of the array such that the portion of the biological sample and the portion of the array contact the reagent medium, the spacer is disposed between the first substrate and the second substrate and is configured to maintain the reagent medium within a chamber formed by the first substrate, the second substrate, and the spacer, and to maintain a separation distance between the first substrate and the second substrate, where the spacer is positioned to surround an area on the first substrate on which the biological sample is disposed and/or the array disposed on the second substrate, where the area of the first substrate, the spacer, and the second substrate at least partially encloses a volume including the biological sample.

In some embodiments, the releasing in step (d) includes contacting the biological sample with a reagent medium including a permeabilization agent and an agent for releasing the ligation product, thereby permeabilizing the biological sample and releasing the ligation product from the nucleic acid analyte.

In some embodiments, the agent for releasing the ligation product includes a nuclease. In some embodiments, the permeabilization agent includes a protease (e.g., trypsin, pepsin, elastase, or proteinase K).

In some embodiments, the method includes determining (i) all or a part of the sequence of the ligation product, or a complement thereof, and (ii) the spatial barcode, or a complement thereof, and using the determined sequence of (i) and (ii) to determine a location of the nucleic acid analyte in the biological sample.

In some embodiments, the determining includes sequencing (i) all or a part of the sequence of the ligation product, or a complement thereof, and (ii) the sequence of the spatial barcode, or a complement thereof.

In some embodiments, the capture probe, the first capture probe, and/or the second capture probe, if present, includes a poly(T) sequence, one or more functional domains, a unique molecular identifier, a cleavage domain, or combinations thereof.

Also provided herein are methods for mitigating mislocalization of target analytes captured on a spatial array, the method including: (a) providing a biological sample on the spatial array including a plurality of capture probes, where a capture probe of the plurality of capture probes includes: (i) a spatial barcode and (ii) a capture moiety; and (b) covalently reacting the capture moiety of the capture probe with a reactive moiety of a ligation product to form a captured ligation product, thereby mitigating mislocalization of the captured ligation product and its subsequent mislocalization on the spatial array, where the ligation product includes a sequence that is substantially complementary to a sequence of a target analyte in the biological sample, and where the capture moiety and the reactive moiety react together to form a junction configured for replication by a polymerase.

Also provided herein are methods for mitigating mislocalization of target analytes captured on a spatial array, the method including: (a) providing a biological sample on the spatial array including a plurality of capture probes, where a capture probe of the plurality of capture probes includes: (i) a spatial barcode and (ii) a capture moiety, or where a first capture probe of the plurality of capture probes includes (i) a spatial barcode and where a second capture probe of the plurality of capture probes includes (ii) a capture moiety; and (b) covalently reacting the capture moiety of the capture probe with a reactive moiety of a ligation product to form a captured ligation product, thereby mitigating mislocalization of the captured ligation product and its subsequent mislocalization on the spatial array, where the ligation product includes a sequence that is substantially complementary to a sequence of an analyte in the biological sample, and where the capture moiety of the capture probe and the reactive moiety react together to form a junction that is not configured for replication by a polymerase.

In some embodiments, migration of the captured ligation product includes vertical migration between the biological sample and the spatial array.

Also provided herein are methods for increasing sensitivity of a spatial array, the method including: (a) providing a biological sample on the spatial array including a plurality of capture probes, where a capture probe of the plurality of capture probes includes: (i) a spatial barcode and (ii) a capture moiety; and (b) covalently reacting the capture moiety of the capture probe with a reactive moiety of a ligation product to form a captured ligation product, thereby increasing sensitivity of the spatial array, as compared to an array lacking the capture moiety, where the ligation product includes a sequence that is substantially complementary to a sequence of a target analyte in the biological sample, and where the capture moiety and the reactive moiety react together to form a junction configured for replication by a polymerase.

Also provided herein are methods for increasing sensitivity of a spatial array, the method including: (a) providing a biological sample on the spatial array including a plurality of capture probes, where a capture probe of the plurality of capture probes includes: (i) a spatial barcode and (ii) a capture moiety, or where a first capture probe of the plurality of capture probes includes (i) a spatial barcode and where a second capture probe of the plurality of capture probes includes (ii) a capture moiety; and (b) covalently reacting the capture moiety of the capture probe with a reactive moiety of a ligation product to form a captured ligation product, thereby increasing sensitivity of the spatial array, as compared to an array lacking the capture moiety, where the ligation product includes a sequence that is substantially complementary to a sequence of an analyte in the biological sample, and where the capture moiety and the reactive moiety react together to form a junction that is not configured for replication by a polymerase.

Also provided herein are methods for decreasing loss of target analytes from a biological sample on a spatial array, the method including: (a) providing the biological sample on the spatial array including a plurality of capture probes, where a capture probe of the plurality of capture probes includes: (i) a spatial barcode and (ii) a capture moiety; and (b) covalently reacting the capture moiety with a reactive moiety of a ligation product to form a captured ligation product, thereby decreasing loss of target analytes from the biological sample on the spatial array, as compared to an array lacking the capture moiety, where the ligation product includes a sequence that is substantially complementary to a sequence of a target analyte in the biological sample, and where the capture moiety and the reactive moiety react together to form a junction configured for replication by a polymerase.

Also provided herein are methods for decreasing loss of target analytes from a biological sample on a spatial array, the method including: (a) providing the biological sample on the spatial array including a plurality of capture probes, where a capture probe of the plurality of capture probes includes: (i) a spatial barcode and (ii) a capture moiety, or where a first capture probe of the plurality of capture probes includes (i) a spatial barcode and where a second capture probe of the plurality of capture probes includes (ii) a capture moiety; and (b) covalently reacting the capture moiety with a reactive moiety of a ligation product to form a captured ligation product, thereby decreasing loss of target analytes from the biological sample on the spatial array, as compared to an array lacking the capture moiety, where the ligation product includes a sequence that is substantially complementary to a sequence of an analyte in the biological sample, and where the capture moiety and the reactive moiety react together to form a junction that is not configured for replication by a polymerase.

Also provided herein are methods for improving binding of target analytes to a spatial array, the method including: (a) providing a biological sample on the spatial array including a plurality of capture probes, where a capture probe of the plurality of capture probes includes: (i) a spatial barcode and (ii) a capture moiety; and (b) covalently reacting the capture moiety of the capture probe with a reactive moiety of a ligation product to form a captured ligation product, thereby improving binding of target analytes to the spatial array, as compared to an array lacking the capture moiety, where the ligation product includes a sequence that is substantially complementary to a sequence of a target analyte in the biological sample, and where the capture moiety and the reactive moiety react together to form a junction configured for replication by a polymerase.

Also provided herein are methods for improving binding of target analytes to a spatial array, the method including: (a) providing a biological sample on the spatial array including a plurality of capture probes, where a capture probe of the plurality of capture probes includes: (i) a spatial barcode and (ii) a capture moiety, or where a first capture probe of the plurality of capture probes includes (i) a spatial barcode and where a second capture probe of the plurality of capture probes includes (ii) a capture moiety; and (b) covalently reacting the capture moiety with a reactive moiety of a ligation product to form a captured ligation product, thereby improving binding of target analytes to the spatial array, as compared to an array lacking the capture moiety, where the ligation product includes a sequence that is substantially complementary to a sequence of an analyte in the biological sample, and where the capture moiety and the reactive moiety react together to form a junction that is not configured for replication by a polymerase.

In some embodiments, the capture probe, the first capture probe, or the second capture probe, if present, is positioned with its 3′ end or its 5′ end distal to a surface of the array. In some embodiments, the capture moiety is distal to the surface of the array. In some embodiments, the capture moiety is positioned at the 3′ end of the capture probe, and where the reactive moiety is positioned on the 5′ end of the ligation product. In some embodiments, the capture moiety is positioned at the 5′ end of the capture probe, where the reactive moiety is positioned on the 5′ end of the ligation product.

In some embodiments, the capture probe includes a priming region at its 3′ end. In some embodiments, the priming region is directly adjacent to the capture moiety.

In some embodiments, the capture probe or the first capture probe includes a cleavable moiety disposed between a surface of the array and the spatial barcode. In some embodiments, the capture probe or the first capture probe includes a priming region and a cleavable moiety disposed in proximity to the priming region. In some embodiments, the cleavable moiety includes a cleavage domain (e.g., disulfide linker, sequence recognized and cleaved by a uracil-DNA glycosylase, an apurinic/apyrimidinic (AP) endonuclease (APEI), a uracil-specific excision reagent (USER), an endonuclease VIII, etc.).

In some embodiments, the capture moiety and/or the reactive moiety includes an azide moiety, an alkyne moiety, a phosphorothioate moiety, a leaving moiety (e.g., an iodide), a carboxyl moiety (e.g., —CO₂H), an amino moiety (e.g., —NR₁R₂), a phosphate moiety, a thiol moiety, streptavidin, avidin, or biotin.

In some embodiments, the capture moiety and the reactive moiety react together to form a junction, and where the junction includes a click signature linkage (e.g., SPAAC [azide moiety and cyclooctyne moiety] or Diels-Alder [diene+dienophile, such as maleimide and furan], which may not be read-through by a polymerase), a triazole linkage, an isoxazoline linkage, an S-phosphorothioester linkage, a phosphorothioate linkage, an amide linkage, a disulfide linkage, a phosphoramidate linkage, a streptavidin/biotin linkage, or an avidin/biotin linkage.

Also provided herein are spatial arrays including: a plurality of capture probes, where a capture probe of the plurality of capture probes includes: (i) a spatial barcode and (ii) a capture moiety, and where the capture moiety of the capture probe is configured to covalently react with a reactive moiety of a templated ligation probe to form a junction configured for replication by a polymerase, and where the templated ligation probe includes a sequence that is complementary to a sequence of an analyte in a biological sample.

Also provided herein are spatial arrays including: a plurality of capture probes, where a capture probe of the plurality of capture probes includes (i) a spatial barcode and (ii) a capture moiety, or where a first capture probe of the plurality of capture probes includes (i) a spatial barcode and where a second capture probe of the plurality of capture probes includes (ii) a capture moiety, where the capture moiety is configured to covalently react with a reactive moiety of a templated ligation probe to form a junction that is not configured for replication by a polymerase, and where the templated ligation probe includes a sequence that is complementary to a sequence of an analyte in a biological sample.

Also provided herein are spatial arrays including: a plurality of capture probes, where a capture probe of the plurality of capture probes includes: (i) a spatial barcode and (ii) a capture moiety, and where the capture moiety is configured to covalently react with a reactive moiety of a ligation product to form a junction configured for replication by a polymerase, and where the ligation product includes a sequence that is substantially complementary to a sequence of an analyte in a biological sample.

Also provided herein are spatial arrays including: a plurality of capture probes, where a capture probe of the plurality of capture probes includes (i) a spatial barcode and (ii) a capture moiety, or where a first capture probe of the plurality of capture probes includes (i) a spatial barcode and where a second capture probe of the plurality of capture probes includes (ii) a capture moiety, where the capture moiety is configured to covalently react with a reactive moiety of a ligation product to form a junction that is not configured for replication by a polymerase, and where the ligation product includes a sequence that is substantially complementary to a sequence of an analyte in a biological sample.

In some embodiments, the ligation product is covalently bound to the capture moiety of the capture probe or the capture moiety of the second capture probe, if present.

In some embodiments, the capture probe, the first capture probe, or the second capture probe, if present, is positioned with its 3′ end or its 5′ end distal to a surface of the spatial array. In some embodiments, the capture moiety is distal to a surface of the spatial array. In some embodiments, the capture moiety is positioned at the 3′ end of the capture probe, and where the reactive moiety is positioned on the 5′ end of the ligation product or the templated ligation probe, if present.

In some embodiments, the capture moiety is positioned at the 5′ end of the capture probe, where the reactive moiety is positioned on the 5′ end of the ligation product or the templated ligation probe, if present.

In some embodiments, the capture probe includes a priming region at its 3′ end. In some embodiments, the priming region is directly adjacent of the capture moiety.

In some embodiments, the capture probe or the first capture probe includes a cleavable moiety disposed between a surface of the spatial array and the spatial barcode. In some embodiments, the capture probe or the first capture probe includes a priming region and a cleavable moiety disposed in proximity to the priming region.

In some embodiments, the cleavable moiety includes a cleavage domain (e.g., disulfide linker, sequence recognized and cleaved by a uracil-DNA glycosylase, an apurinic/apyrimidinic (AP) endonuclease (APEI), a uracil-specific excision reagent (USER), an endonuclease VIII, or any described herein).

In some embodiments, the capture moiety and/or the reactive moiety includes an azide moiety, an alkyne moiety, a phosphorothioate moiety, a leaving moiety (e.g., an iodide), a carboxyl moiety (e.g., —CO₂H), an amino moiety (e.g., —NR₁R₂, in which each of R₁ and R₂ is, independently, hydrogen (H) or alkyl), a phosphate moiety, a thiol moiety, streptavidin, avidin, or biotin.

In some embodiments, the capture moiety and the reactive moiety react together to form a junction, and where the junction includes a click signature linkage, a triazole linkage, an isoxazoline linkage, an S-phosphorothioester linkage, a phosphorothioate linkage, an amide linkage, a disulfide linkage, a phosphoramidate linkage, a streptavidin/biotin linkage, or an avidin/biotin linkage.

Also provided herein are kits including: a substrate including a plurality of capture probes, where a capture probe of the plurality of capture probes includes: (i) a spatial barcode and (ii) a capture moiety; a plurality of templated ligation probes comprising a first probe and second probe, where a templated ligation probe of the plurality of templated ligation probes includes a reactive moiety; where the capture moiety is configured to covalently react with the reactive moiety to form a junction configured for replication by a polymerase, and where the templated ligation probe includes a sequence that is complementary to a sequence of an analyte in a biological sample.

Also provided herein are kits including: a substrate including a plurality of capture probes, where a capture probe of the plurality of capture probes includes (i) a spatial barcode and (ii) a capture moiety, or where a first capture probe of the plurality of capture probes includes (i) a spatial barcode and where a second capture probe of the plurality of capture probes includes (ii) a capture moiety; a plurality of templated ligation probes comprising a first probe and second probe, where a templated ligation probe of the plurality of templated ligation probes includes a reactive moiety; where the capture moiety is configured to covalently react with the reactive moiety to form a junction that is not configured for replication by a polymerase, and where the templated ligation probe includes a sequences that is complementary to sequence of an analyte in a biological sample.

In some embodiments, the kit includes instructions for performing any of the methods described herein.

In some embodiments, the capture probe, the first capture probe, and/or the second capture probe, if present, includes a cleavable moiety.

In some embodiments, each of a pair of templated ligation probes includes a sequence that is complementary to adjacent or non-adjacent sequences of an analyte.

In some embodiments, the substrate includes any of the spatial arrays described herein.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate, including a biological sample, and a second substrate are brought into proximity with one another.

FIG. 1B shows a fully formed sandwich configuration creating a chamber formed from the one or more spacers, the first substrate, and the second substrate.

FIG. 2A shows a perspective view of an example sample handling apparatus in a closed position.

FIG. 2B shows a perspective view of the example sample handling apparatus of FIG. 2A in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with the spacer contacting both the first substrate and the second substrate.

FIG. 4A shows a side view of the angled closure workflow.

FIG. 4B shows a top view of the angled closure workflow.

FIG. 5 shows an exemplary barcoded capture probe as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows and exemplary workflow for performing templated capture of a target analyte resulting in a ligation product, and FIG. 9B shows an exemplary workflow for capturing a ligation product from FIG. 9A on a substrate.

FIG. 10 is a schematic diagram showing an exemplary workflow to obtain a captured ligation product using a capture probe having a barcode and a capture moiety.

FIGS. 11A-11D show various approaches for chemically-mediated nucleic acid reactions. FIG. 11A illustrates formation of non-limiting triazole bonds. FIG. 11B illustrates formation of non-limiting phosphoramidite or phosphorothioate bonds having thiol linkages.

FIG. 11C illustrates formation of non-limiting aldehyde bonds. FIG. 11D illustrates formation of non-limiting reversible bonds.

FIGS. 12A-12E show various approaches for chemically-mediated nucleic acid ligation. FIG. 12A illustrates formation of a non-limiting triazole bond. FIG. 12B illustrates formation of a non-limiting phosphorothioate bond. FIG. 12C illustrates formation of a non-limiting amide bond. FIG. 12D illustrates a formation of a non-limiting phosphoramidate bond. FIG. 12E illustrates a non-limiting conjugation reaction.

FIG. 13 is a schematic diagram showing an exemplary workflow to provide a junction between a ligation product and a capture probe having a barcode.

FIG. 14 is a schematic diagram showing an exemplary workflow to obtain a captured ligation product using a capture probe having a barcode, a priming region (e.g., oligo-dT), a capture moiety, and a cleavable moiety in proximity to the capture moiety.

FIG. 15 is a schematic diagram showing an exemplary workflow to obtain a captured ligation product using a first capture probe having a barcode and a priming region (e.g., oligo-dT) and a second capture probe (e.g., capture oligo) having a capture moiety.

### DETAILED DESCRIPTION

Described herein are methods and compositions that may be used to minimize target (e.g., transcript or molecule) mislocalization. Without wishing to be limited by mechanism, capture chemistry is proposed to more effectively attach a target sequence to the capture probe, thereby potentially controlling migration of target sequences. In some embodiments, the target sequence can be an intermediate agent (e.g., a ligation product, such as any described herein) that is a proxy for an analyte.

### A. Spatial Analysis

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample. Intermediate agents (e.g., ligation products or other sequences) can serve as proxies of target analytes in the methods and compositions herein.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363 (6434): 1463-1467, 2019; Lee et al., Nat. Protoc. 10 (3): 442-458, 2015; Trejo et al., PLOS ONE 14 (2): e0212031, 2019; Chen et al., Science 348 (6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I) (b) of PCT Publication No. WO2020/176788 and/or U.S. Patent Application Publication No. 2020/0277663. Typically, a “barcode” is a label, or identifier, which conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of nucleic acid analytes include, but are not limited to, DNA, RNA (e.g., mRNA), and combinations thereof. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I) (c) of PCT Publication No. WO2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

The biological sample as used herein can be any suitable biological sample described herein or known in the art. In some embodiments, the biological sample is a tissue. In some embodiments, the tissue sample is a solid tissue sample. In some embodiments, the biological sample is a tissue section. In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed using cryosectioning. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plants, an insect, an arachnid, a nematode (e.g., Caenorhabditis elegans), a fungi, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, the biological sample is not fixed with paraformaldehyde (PFA). In some instances, when the biological sample is fixed with a fixative including an alcohol (e.g., methanol or acetone-methanol mixture), it is not decrosslinked afterward. In some preferred embodiments, the biological sample is fixed with a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some embodiments when a fixed frozen tissue sample is treated with a sucrose gradient, it can be rehydrated with an ethanol gradient. In some embodiments, the PFA or formalin fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used for antigen retrieval to decrosslink antigens and fixation medium in the biological sample. Thus, any suitable decrosslinking agent can be used in addition to or alternatively to citrate buffer. In some embodiments, for example, the biological sample (e.g., a fixed frozen tissue sample) is decrosslinked with TE buffer.

In any of the foregoing, the biological sample can further be stained, imaged, and/or destained. For example, in some embodiments, a fresh frozen tissue sample or fixed frozen tissue sample is stained (e.g., via cosin and/or hematoxylin), imaged, destained (e.g., via HCl), or a combination thereof. In some embodiments, when a fresh frozen tissue sample is fixed in methanol, it is treated with isopropanol prior to being stained (e.g., via cosin and/or hematoxylin), imaged, destained (e.g., via HCl), or a combination thereof. In some embodiments when a fixed frozen tissue sample is treated with a sucrose gradient, it can be rehydrated with an ethanol gradient before being stained, (e.g., via cosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9 (10): 5188-96; Kap M. et al., PLOS One.; 6 (11): c27704 (2011); and Mathieson W. et al., Am J Clin Pathol.; 146 (1): 25-40 (2016), each of which are hereby incorporated by reference in their entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Non-limiting examples of stains include histological stains (e.g., hematoxylin and/or cosin) and immunological stains (e.g., fluorescent stains). The biological sample can be stained using Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. In some instances, PAS staining is performed after formalin or acetone fixation. In some embodiments, a biological sample (e.g., a fixed and/or stained biological sample) can be imaged. Biological samples are also described in Section (I) (d) of PCT Publication No. WO2020/176788 and/or U.S. Patent Application Publication No. 2020/0277663.

The following embodiments can be used with any of the methods described herein. In some embodiments, the biological sample is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding the primer to the biological sample.

In some embodiments, the method includes staining the biological sample. In some embodiments, the staining includes the use of hematoxylin and/or cosin. In some embodiments, a biological sample can be stained using any number of biological stains, including but not limited to, acridine orange, Bismarck brown, carmine, coomassie blue, cresyl violet, DAPI, cosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I) (d) (ii) (13) or the Exemplary Embodiments Section of PCT Publication No. WO2020/176788 and/or U.S. Patent Application Publication No. 2020/0277663. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, the entire contents of which are incorporated herein by reference.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II) (b) (e.g., subsections (i)-(vi)) of PCT Publication No. WO2020/176788 and/or U.S. Patent Application Publication No. 2020/0277663. Generation of capture probes can be achieved by any appropriate method, including those described in Section (II) (d) (ii) of PCT Publication No. WO2020/176788 and/or U.S. Patent Application Publication No. 2020/0277663.

In some instances, a capture probe and a nucleic acid analyte (or any other nucleic acid to nucleic acid interaction) occurs because the sequences of the two nucleic acids are substantially complementary to one another. By “substantial,” “substantially” and the like, two nucleic acid sequences can be complementary when at least 60% of the nucleotide residues of one nucleic acid sequence are complementary to nucleotide residues in the other nucleic acid sequence. The complementary residues within a particular complementary nucleic acid sequence need not always be contiguous with each other and can be interrupted by one or more non-complementary residues within the complementary nucleic acid sequence. In some embodiments, at least 60%, but less than 100%, of the residues of one of the two complementary nucleic acid sequences are complementary to residues in the other nucleic acid sequence. In some embodiments, at least 70%, 80%, 90%, 95% or 99% of the residues of one nucleic acid sequence are complementary to residues in the other nucleic acid sequence. Sequences are said to be “substantially complementary” when at least 60% (e.g., at least 70%, at least 80%, or at least 90%) of the residues of one nucleic acid sequence are complementary to residues in the other nucleic acid sequence.

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II) (b) (ix) of PCT Publication No. WO2020/176788 and/or Section (II) (b) (viii) U.S. Patent Application Publication No. 2020/0277663.

In some embodiments, the biological sample is mounted on a first substrate and the substrate comprising the array of capture probes is on a second substrate. During this process, one or more analytes or analyte derivatives (e.g., intermediate agents; e.g., ligation products) are released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

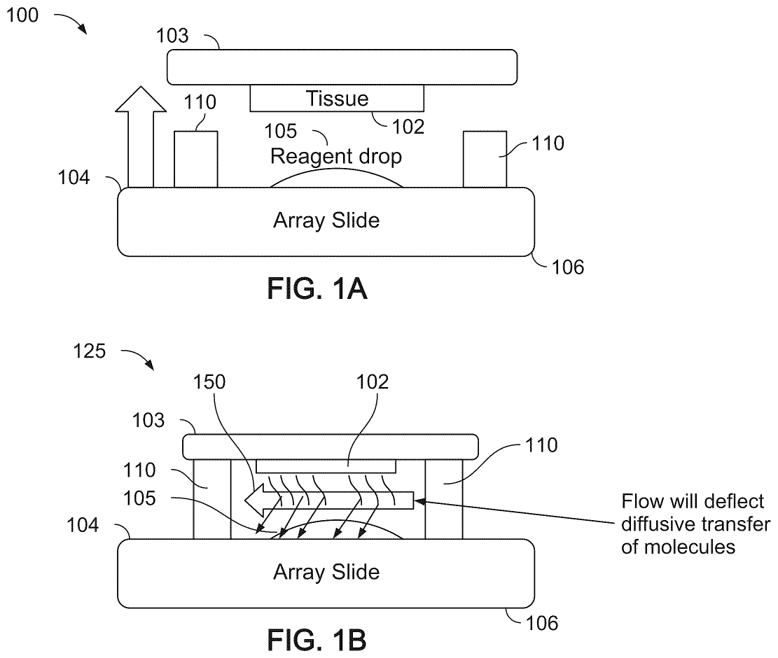

FIG. 1A shows an exemplary sandwiching process 100 where a first substrate (e.g., slide 103), including a biological sample 102 (e.g., a tissue section), and a second substrate (e.g., array slide 104 including an array having spatially barcoded capture probes 106) are brought into proximity with one another. As shown in FIG. 1A a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffusc) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully scaled chamber 150 resulting from the one or more spacers 110, the first substrate, and the second substrate may reduce or prevent flow from undesirable convective movement of transcripts and/or molecules over the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Pub. No. 2021/0189475, and PCT Publ. No. WO 2022/061152 A2, each of which are incorporated by reference in their entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

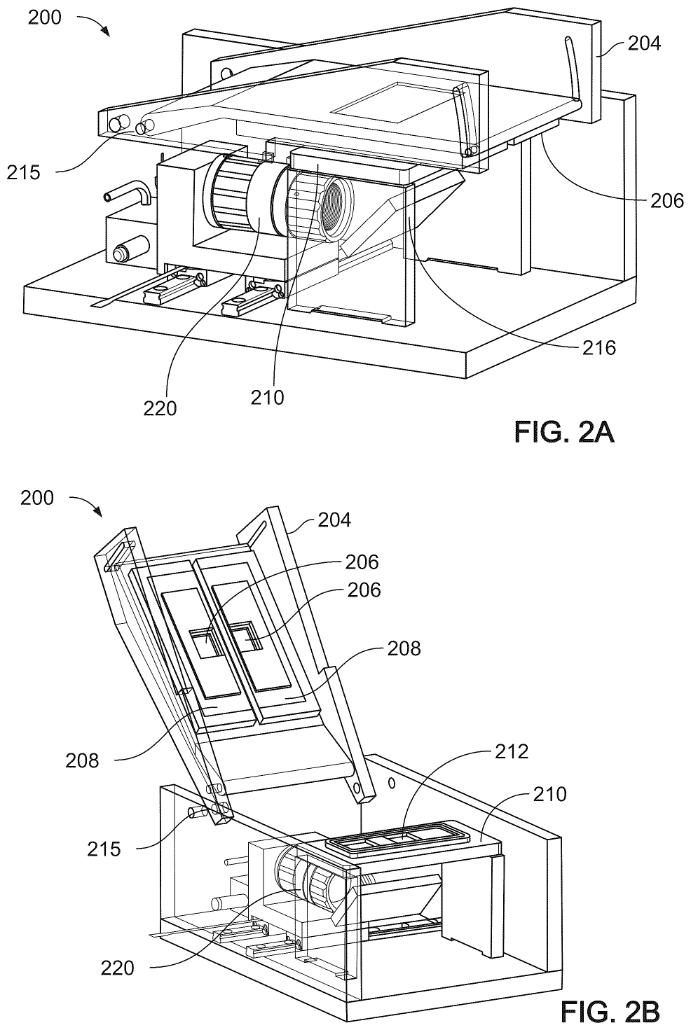

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

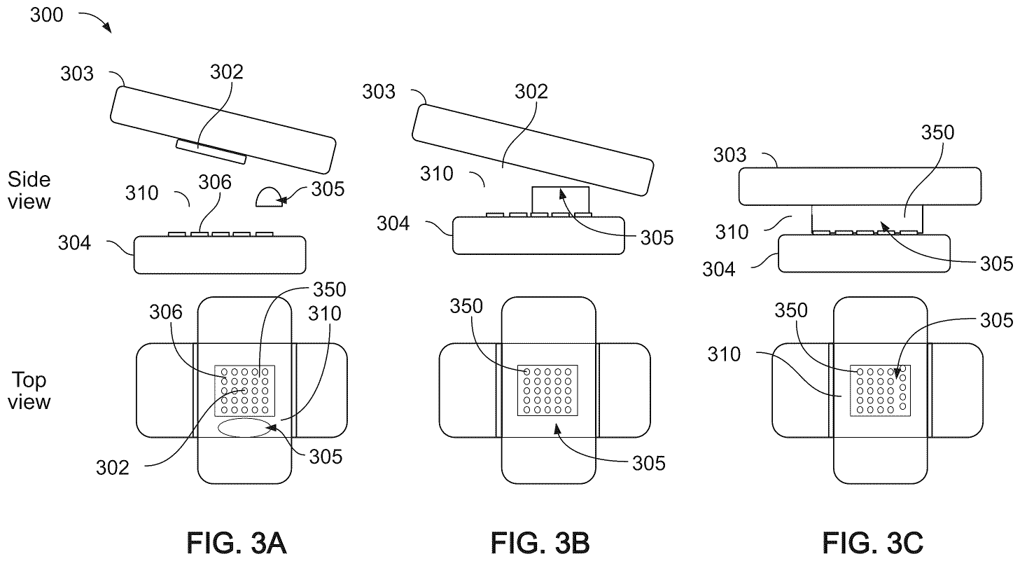

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the second substrate) may contact the reagent medium 305. The dropped side of the first substrate may urge the reagent medium 305 toward the opposite direction (e.g., towards an opposite side of the spacer 310, towards an opposite side of the first substrate relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 forms the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

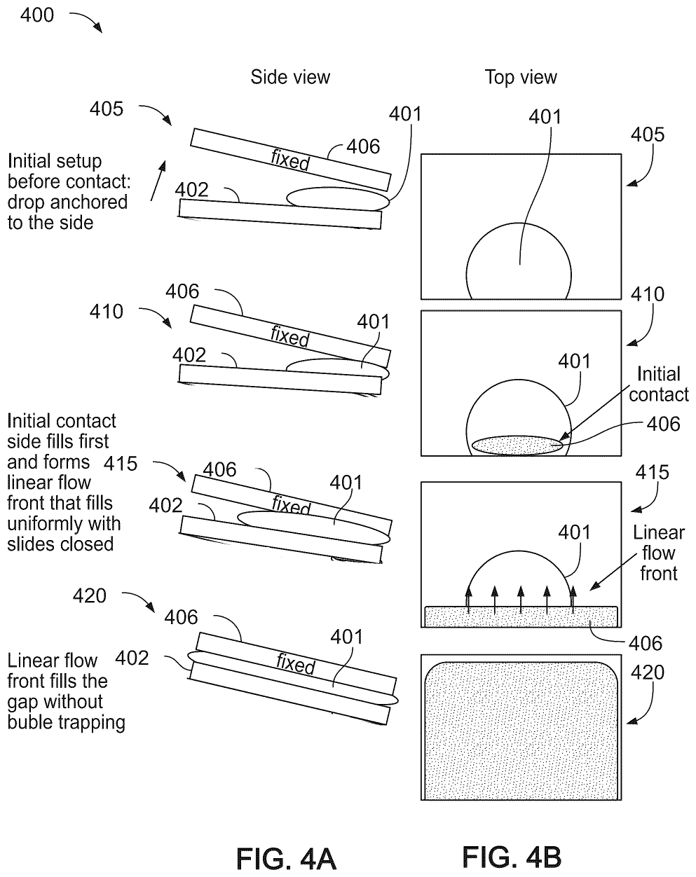

FIG. 4A is a side view of the angled closure workflow 400 in accordance with some exemplary implementations. FIG. 4B is a top view of the angled closure workflow 400 in accordance with some exemplary implementations. As shown at 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side and creating a linear or low curvature flow front that may prevent or reduce bubble trapping between the substrates.

At step 420, the reagent medium 401 fills the gap between the substrate 406 and the substrate 402. The linear flow front of the liquid reagent may form by squeezing the 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening sample holder). Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, pepsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of sodium dodecyl sulfate (SDS) or a sodium salt thereof, proteinase K, pepsin, N-lauroylsarcosine, and RNase.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In certain embodiments a dried permeabilization reagent is applied or formed as a layer on the first substrate or the second substrate or both prior to contacting the biological sample and the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II) (b) (vii) of PCT Publication No. WO2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligation products that can serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture domain and the sequence of the spatial barcode of the capture probe.

In some embodiments, extended capture probes are amplified (e.g., in bulk solution or on the array) to yield quantities that are sufficient for downstream analysis, e.g., via sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II) (a) of PCT Publication No. WO2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II) (g) of PCT Publication No. WO2020/176788 and/or U.S. Patent Application Publication No. 2020/0277663. Some quality control measures are described in Section (II) (h) of PCT Publication No. WO2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and/or 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor or proximity based analysis); determination of up- and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II) (c) of PCT Publication No. WO2020/176788 and/or U.S. Patent Application Publication No. 2020/0277663. Exemplary features and geometric attributes of an array can be found in Sections (II) (d) (i), (II) (d) (iii), and (II) (d) (iv) of PCT Publication No. WO2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II) (c) of PCT Publication No. WO2020/176788 and/or U.S. Patent Application Publication No. 2020/0277663.

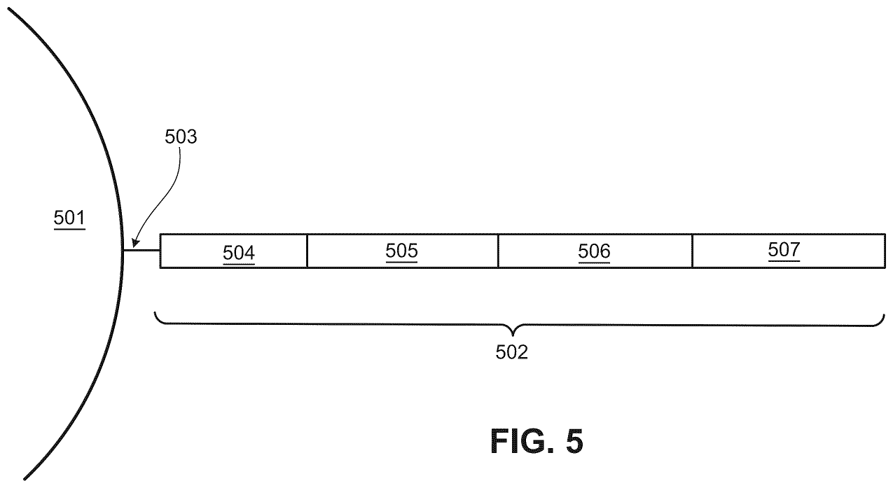

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that are useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

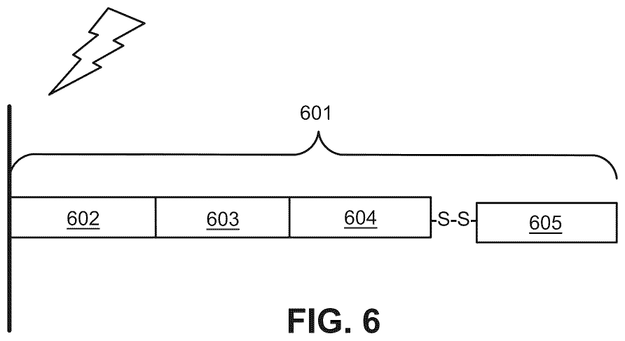

FIG. 6 is a schematic illustrating a cleavable capture probe, wherein the cleaved capture probe can enter into a cell and bind to analytes within the sample. The capture probe 601 contains a cleavage domain 602, a cell penetrating peptide 603, a reporter molecule 604, and a disulfide bond (—S—S—). 605 represents all other parts of a capture probe, for example a spatial barcode and a capture domain.

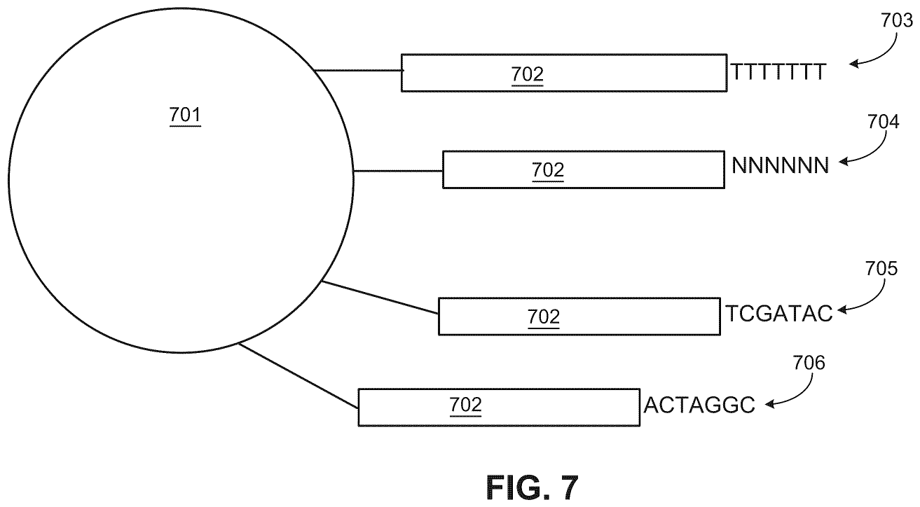

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode but have different capture domains designed to associate the spatial barcode of the feature with more than one target analyte. For example, a feature may be coupled to four different types of spatially-barcoded capture probes, each type of spatially-barcoded capture probe possessing the spatial barcode 702. One type of capture probe associated with the feature includes the spatial barcode 702 in combination with a poly(T) capture domain 703, designed to capture mRNA target analytes. A second type of capture probe associated with the feature includes the spatial barcode 702 in combination with a random N-mer capture domain 704 for gDNA analysis. A third type of capture probe associated with the feature includes the spatial barcode 702 in combination with a capture domain complementary to the analyte capture agent of interest 705. A fourth type of capture probe associated with the feature includes the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. Sec, e.g., Section (II) (b) (e.g., subsections (i)-(vi)) of WO 2020/176788 and/or U.S. Patent Application Publication No. 2020/0277663. Generation of capture probes can be achieved by any appropriate method, including those described in Section (II) (d) (ii) of WO 2020/176788 and/or U.S. Patent Application Publication No. 2020/0277663.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequences 504 is common to all of the probes attached to a given feature. In some embodiments, the UMI sequence 506 of a capture probe attached to a given feature is different from the UMI sequence of a different capture probe attached to the given feature.

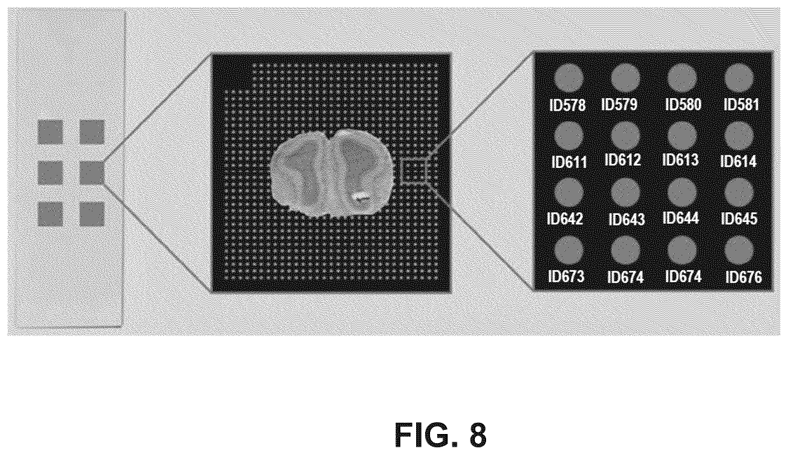

FIG. 8 depicts an exemplary arrangement of barcoded features within an array. From left to right, FIG. 8 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, showing the specific identification of multiple features within the array (labelled as ID578, ID579, ID560, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. Sec, e.g., Credle et al., Nucleic Acids Res. 2017 Aug. 21; 45 (14): c128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., a T4 RNA ligase (Rnl2), a PBCV-1 DNA ligase, a Chlorella virus DNA ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location and optionally the abundance of the analyte in the biological sample.

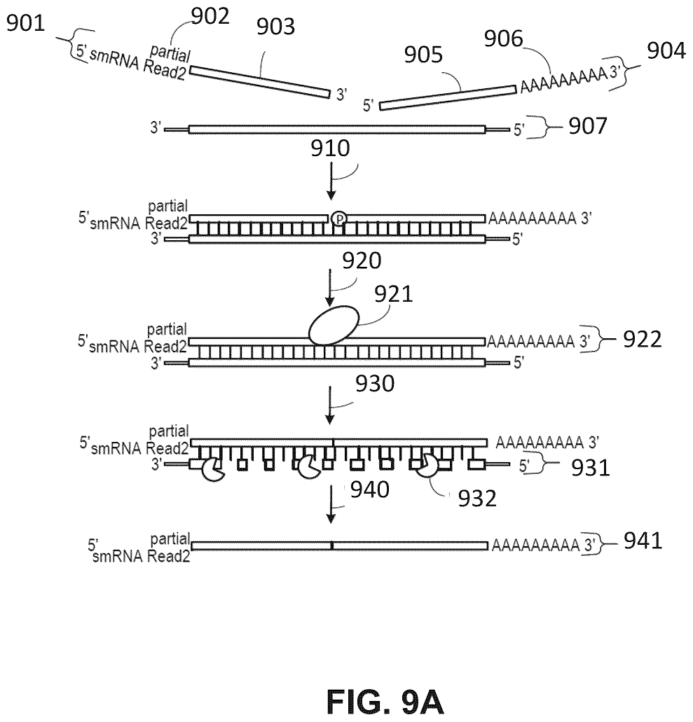

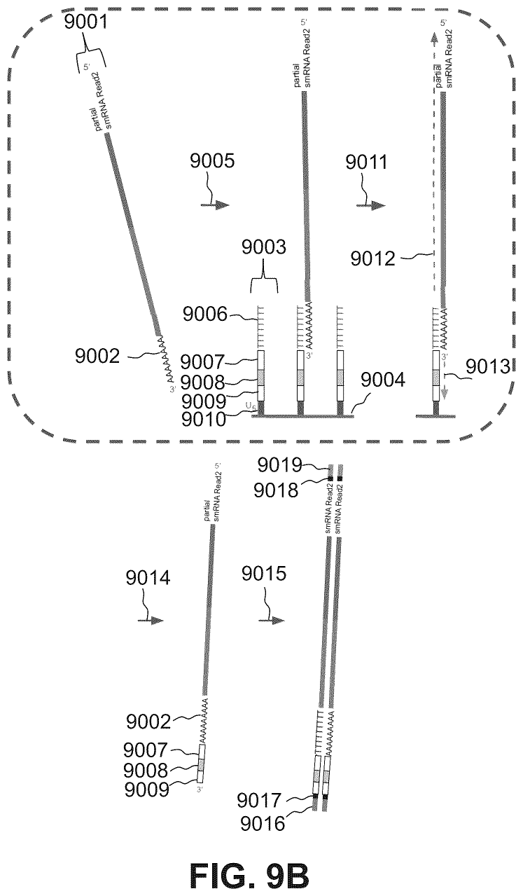

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly-A sequence) 906, the first probe 901 and a second probe 904 hybridize 910 to an analyte 907. A ligase 921 ligates 920 the first probe to the second probe thereby generating a ligation product 922. The ligation product is released 930 from the analyte 931 by digesting the analyte using an endoribonuclease 932. The sample is permeabilized 940 and the ligation product 941 is able to hybridize to a capture probe on the substrate. Methods and composition for spatial detection using templated ligation have been described in PCT Publ. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligation product specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, reverse transcription (RT) reagents can be added to permeabilized biological samples. Incubation with the RT reagents can extend the capture probes 9011 to produce spatially-barcoded full-length cDNA 9012 and 9013 from the captured ligation products (e.g., ligation products).

In some embodiments, the extended ligation products can be denatured 9014 from the capture probe and transferred (e.g., to a clean tube) for downstream processing, such as, amplification, and/or library construction. The spatially-barcoded ligation products can be amplified 9015 via PCR prior to library construction. P5 9016 and P7 9019 can be used as sequences that are complementary to sequencing probes for immobilization of the library on the sequencing flow cell and i5 9017 and i7 9018 can be used as sample indexes. The resulting amplicons can then be sequenced using paired-end sequencing using TruSeq Read 1 and TruSeq Read 2 as sequencing primer sites.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022).

In some embodiments, spatial analysis can be performed using dedicated hardware and/or software, such as any of the systems described in Sections (II) (c) (ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Methods of Capturing a Target Analyte

High-resolution spatial transcriptomics relies, in part, on minimizing target analyte mislocalization. For example, target sequences typically migrate vertically to the substrate comprising an array, where they are captured by the capture domain of a capture probe. However, it may happen that, due to weak association of the target sequence with the capture domain of the capture probe, the target sequence may dissociate from the capture domain and migrate non-vertically (e.g., horizontally to the surface of the substrate) and be captured by the capture domain of a capture probe that does not correspond to the original location in a biological sample. As such, weak binding chemistry can ineffectively localize the target at the initial binding site, thereby introducing errors in accurate spatial localization of that target sequence. Accordingly, in some embodiments, the methods herein employ alternative binding chemistry that may be implemented to more effectively associate a target analyte to the capture domain of a capture probe at the initial capture site. In some embodiments, the target analyte can be an intermediate agent (e.g., a ligation product, such as any described herein), which can optionally serve as a proxy for a target analyte.

Target analytes for spatial transcriptomics arrays can include endogenous mRNAs, which are polyadenylated at the 3′ end with a poly(A) tail. In use, a portion of the poly(A) tail is captured by the oligo-dT capture domain present on a surface of an array. Described herein are capturing chemistries having increased stability, as compared to relying solely on hybridization between the poly(A) tail of the mRNA molecule and the oligo-dT capture domain of the capture probe. In one non-limiting instance, a capturing chemistry herein can be employed with a ligation product (e.g., generated by RNA-templated “RTL” approaches), in which the ligation product (not endogenous mRNAs, but a proxy thereof) is captured by a capture probe. In some embodiments, features of the ligation product (or probes used to generate the ligation product) can be configured to provide complementary chemistries that allow for covalently reacting the ligation product to the capture probe. In some embodiments, the complementary chemistries provide a junction having a stability (e.g., as determined by bond strength, reaction kinetics, and the like) that is greater than interactions arising from oligo-dT/poly(A) hybridization generated from the poly(A) of the ligation product and the oligo-dT capture domain of the capture probe.

As used herein, the term “capture chemistry” is used to encompass one or more capture moieties, reactive moieties, reaction products, linkers, reactions, and conditions that provide a junction (directly or indirectly) between a capture probe and a target, in this instance a ligation product, which is a proxy of an endogenous target analyte (e.g., mRNA). A “capture moiety” can be any functional group capable of capturing (directly or indirectly) an analyte (e.g., an analyte of interest) in a biological sample. In some embodiments, the capture moiety is a nucleophile or an electrophile. The capture moiety can be present in a capture domain of the capture probe. In certain embodiments, the capture domain can include one or more capture moieties. In some embodiments, the capture moiety can capture the ligation product by way of a covalent reaction.

The capture moiety can be present within, or at the 5′ or 3′ end, of a capture probe and can be configured to capture any analyte of interest. In some embodiments, the analyte of interest is an intermediate agent. In some embodiments, the intermediate agent is a ligation product, for example, which is a proxy for a target analyte. In some embodiments, the analyte of interest is a target sequence.

The capture moiety can be attached to a capture probe (e.g., a capture probe or a second capture probe) directly or indirectly (e.g., by way of a linker). For example, in some embodiments, the capture moiety can be attached to the capture probe indirectly by way of a cleavable moiety or by way of a linker having a cleavable moiety. Additional capture moieties, capture probes, and linker are described herein.

A capture probe can include any useful combination of one or more barcodes, capture moieties, capture domains, and/or cleavable moieties, such that the combination provides a probe that can interact with a target sequence. In some embodiments, a capture probe can include a capture moiety. In other embodiments, a capture probe can include a capture moiety and a spatial barcode. In yet other embodiments, a capture probe can include a capture moiety, a spatial barcode, and a cleavable moiety. In some embodiments, a capture probe can include a capture moiety and a cleavable moiety. In any of these, an optional priming region and/or an optional capture domain can be present. In some embodiments, the capture domain can serve as a priming region during extension.

Capture domains can be used to interact with the target sequence, although such interactions may not necessarily provide a covalent attachment. In some embodiments, a capture probe can include a capture domain. In other embodiments, a capture probe can include a capture domain and a spatial barcode. A capture probe having a capture domain can be used in combination with another capture having a capture moiety, in which such a probe pair can be used to interact with and capture the target sequence, such as a ligation product that serves as a proxy of a target analyte. A target sequence can include a synthetic or natural sequence.

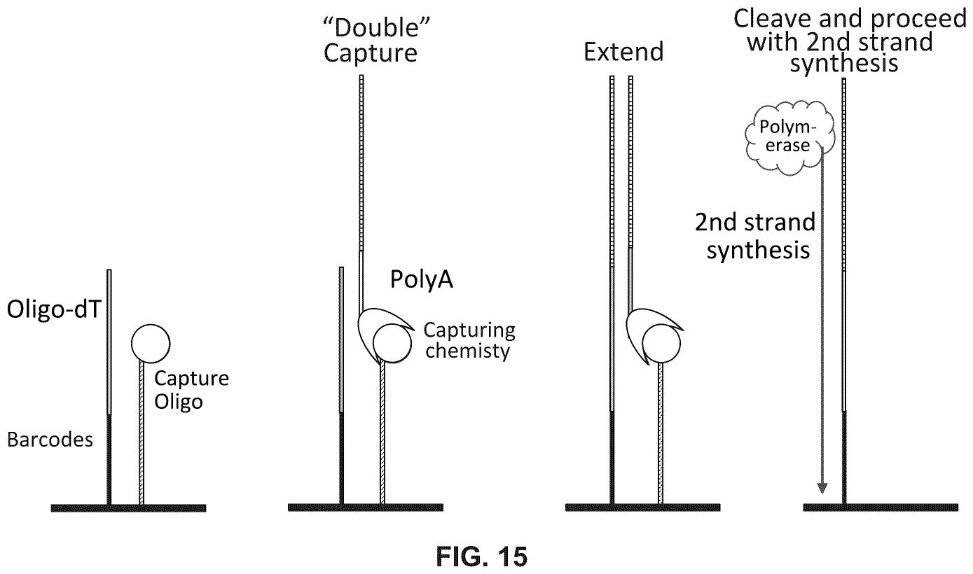

One, two, three, or more capture probes can be employed. In some embodiments, the methods herein can employ an array having a plurality of capture probes, in which a capture probe of the plurality of capture probes can be any probe described herein. In other embodiments, the methods herein can employ an array having a plurality of first capture probes and a plurality of second capture probes, in which the first and second capture probes are different. For instance, the first capture probe can include a spatial barcode, and the second capture probe can include a capture moiety. In use, the first and second capture probes can form a probe pair that interact with one another to capture the target sequence. Such interactions can be such that allow for hybridization between a portion of the first capture probe and a portion of the captured ligation product (a target sequence attached to the second capture probe). FIG. 15 provides a non-limiting example of using a probe pair for dual capture, in which a first capture probe (having a barcode) can be extended to provide a sequence indicative of the target analyte and in which a second capture probe can react with a reactive moiety of the target sequence by way of the capture moiety.

A “reactive moiety” can be any functional group capable of reacting with a capture moiety. In some embodiments, the reactive moiety is a nucleophile or an electrophile. The reactive moiety can be present in the capture probe capture domain of a ligation product or in the capture probe capture domain of a second probe used to generate a ligation product. In certain embodiments, the capture probe capture domain can include one or more reactive moieties. Additional reactive moieties, RTL probes (e.g., first probes, second probes, etc.), and ligation products are described herein.

The capture moiety and the reactive moiety can be selected to provide complementary chemistries that result in the formation of a junction. In turn, the junction can provide a covalent bond between the capture probe and the ligation product. For instance, if the capture moiety includes a nucleophile, then the reactive moiety can include an electrophile that can react with the nucleophile under provided reaction conditions to provide a covalent bond. Useful reaction conditions (e.g., to decrease reaction time, increase reaction efficiency, and the like) can be implemented.

In some embodiments, the capture chemistry can provide a junction that can be replicated, copied, extended, amplified, transcribed, or otherwise processed by a polymerase (e.g., a DNA polymerase, an RNA polymerase, as well as variants or modified forms thereof). In other embodiments, the capture chemistry captures the ligation product (thereby providing a captured ligation product) but provides a junction that cannot be replicated by a polymerase. Instead, the captured ligation product can be further processed to provide a further product that can then undergo downstream analyses (e.g., second strand synthesis, sequencing, etc.).

In one embodiment, described herein are methods for capturing a ligation product, the method comprising: (a) providing a biological sample on an array comprising a plurality of capture probes; and (b) covalently reacting the capture moiety with a reactive moiety of a ligation product to form a captured ligation product. In some embodiments, a capture probe of the plurality of capture probes comprises: (i) a spatial barcode and (ii) a capture moiety. In further embodiments, the capture moiety and the reactive moiety react together to form a junction configured for replication by a polymerase.

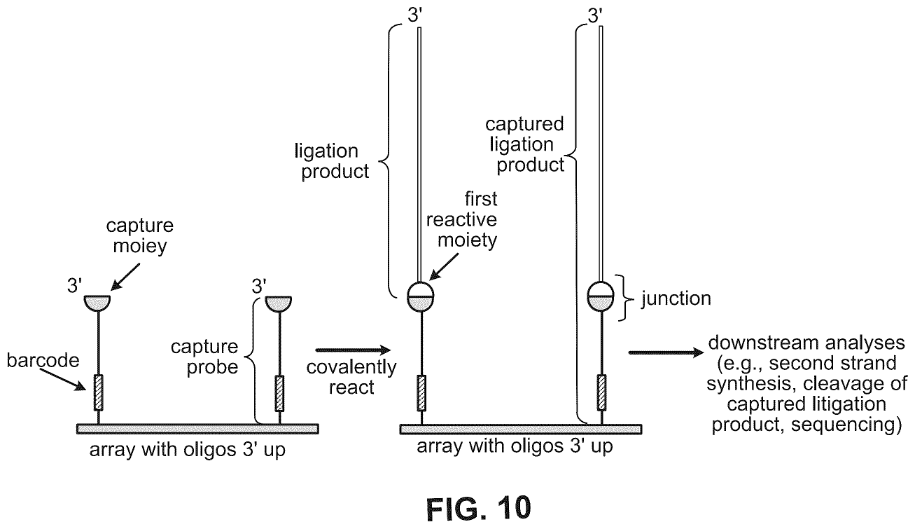

FIG. 10 provides a non-limiting workflow of a method for capturing a ligation product. The array includes a plurality of capture probes, in which at least one capture probe includes: (i) a spatial barcode and (ii) a capture moiety. While the array in FIG. 10 shows the capture probe having its 3′ end to be distal to a surface of the array, other orientations are possible. For instance, the capture probe can be attached to the surface of the array to provide its 5′ end to be distal to a surface.

As seen in FIG. 10, the capture probe can be covalently reacted with a ligation product, in which the capture moiety of the capture probe is reacted with the first reactive moiety of the ligation product to produce a junction. By forming a junction, a more stable capture probe/ligation product complex can be generated, which mitigates unwanted release of the capture ligation product and its subsequent mislocalization on the spatial array. This captured ligation product can undergo further downstream analyses, e.g., second strand synthesis, cleavage of the captured ligation product, sequencing, as well as other processes described herein.

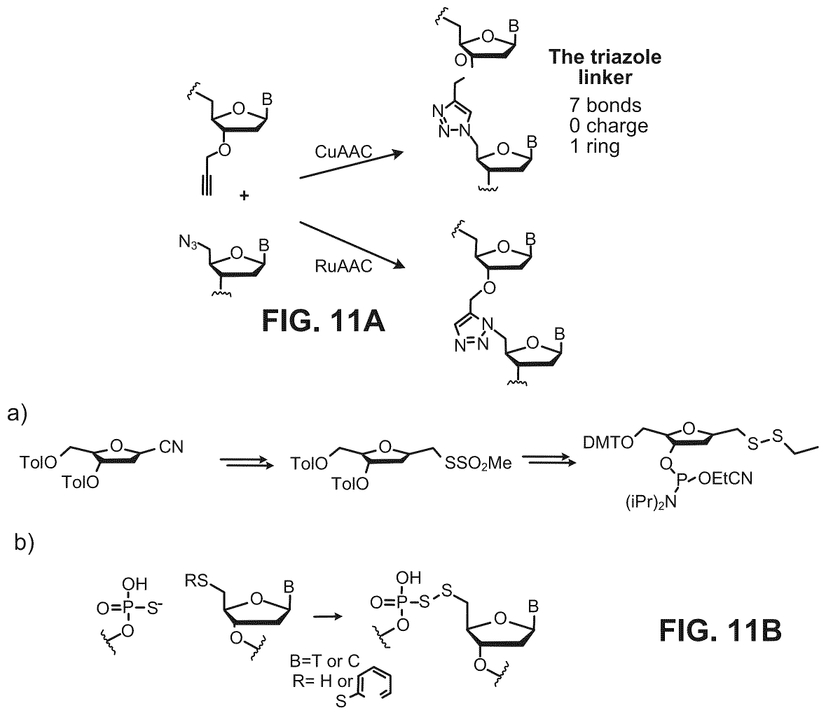

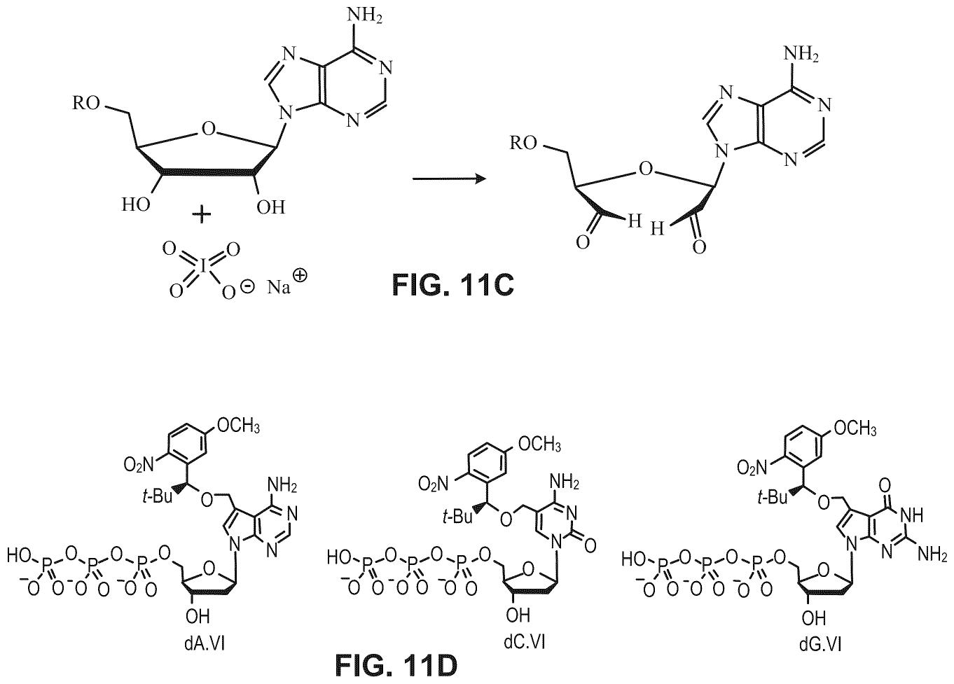

In some embodiments, the junction is configured for replication by a polymerase. FIGS. 11A-11D exemplifies representative reactions. FIG. 11A shows a chemical ligation reaction of an alkyne moiety and an azide moiety reacting under copper-mediated cycloaddition to form a triazole linkage. Copper-free cycloaddition reactions (e.g., ruthenium-mediated cycloaddition) can be employed as well. Recent advances in triazole linkages have shown that a wide variety of polymerases can copy through the non-standard backbone. In one embodiment, the capture probe can be configured to have a capture moiety that is a 3′ alkynyl group (e.g., a C₂₋₁₀ alkynyl group), and the first or second probes (for generating a ligation product) can be configured to have a reactive moiety that is a 5′ azido group (e.g., an —N₃ group). In a particular embodiment, the capture probe can be reacted with a terminal deoxynucleotidyl transferase (TdT) to label the ends of the array with modified bases (e.g., 3′ alkynyl-containing bases). Upon reaction, the junction could provide a continuous 5′-+3′ linkage within the captured ligation product. Other configurations can be implemented to provide other linkages (e.g., a capture moiety that is a 5′ alkynyl group, and a reactive moiety that is a 3′ azido group). These, and other, click-chemistry reactions can be employed to provide a junction having a click signature linkage.

FIG. 11B illustrates formation of non-limiting phosphoramidite or phosphorothioate bonds having thiol linkages. Such phosphoramidite or phosphorothioate chemistry can be employed to modify terminal nucleotides on capture probes and/or ligation products, as well as provide thiol linkages.

FIG. 11C illustrates formation of non-limiting aldehyde bonds. An oxidizing agent can be used to react with diol groups that are present on RNA. For instance, sodium meta-periodate can be used to cleave the C—C bond between diol groups on the terminal ribose, thereby forming aldehyde groups. Aldehyde groups can then be reacted with a nucleophile (e.g., an amino group, such as a primary amino group). Thus, a non-limiting complementary chemistry can include an aldehyde group (e.g., —C(O) H) and an amino group (e.g., —NH₂), and the capture moiety can include the aldehyde and the reactive moiety can include the amino (or vice versa). In one embodiment, the RTL probes employed to form a ligation product can be selected to provide a ligation product having a 5′ terminated with a primary amino group, and the capture probe can be enzymatically terminated with an RNA base and then reacted with sodium meta-periodate to provide a 3′ terminated with an aldehyde group. Alternatively, a reagent can be used to provide a primary amino group at a 5′ end of a ligation product or an mRNA molecule, and the 5′-amino termination ligation product or the 5′-amino terminated mRNA molecule can be provided in the presence of a capture probe having a 3′-aldehyde group. Some polymerases have been shown to polymerize through this backbone linkage.

As can be seen, capture moieties and reactive moieties can be provided in any useful manner. In one non-limiting instance, capture probes are synthesized or prepared to include capture moieties, and then the capture probes are immobilized on a surface of the substrate to generate a spatial array. In another instance, the array includes a plurality of capture probes attached to a surface of a substrate, and terminal modifications are provided to the attached capture probes, thereby providing capture moieties. Accordingly, any methods herein can include (e.g., after providing an array): converting a terminal nucleotide of the capture probe to provide the capture moiety. Such converting can include installing a nucleotide (e.g., a ribose, a ribonucleotide, a modified base, such by enzymatic termination or chemical modification), oxidizing a terminal nucleotide (e.g., a terminal RNA base), ligating a functional oligonucleotide (e.g., to introduce a common feature, reactive group, or moiety to capture probes), or otherwise reacting a terminal nucleotide (e.g., a ribose, a ribonucleotide, a reversibly terminated nucleotide, or a reversibly terminated ribonucleotide of a capture probe).

Furthermore, the RTL probes employed to form a ligation product (e.g., first probes, second probes, and the like) can be synthesized or prepared to include reactive moieties, and then the probes are hybridized to the target analyte to form the ligation product. Alternatively, the ligation product can be formed and then terminally modified to provide the reactive moiety. Accordingly, any methods herein can include (e.g., after forming a ligation product): converting a terminal nucleotide of the ligation product to provide the reactive moiety. Such converting can include installing a nucleotide (e.g., a ribose, a ribonucleotide, a modified base, such by enzymatic termination or chemical modification), oxidizing a terminal nucleotide (e.g., a terminal RNA base), ligating a functional oligonucleotide (e.g., to introduce a common feature, reactive group, or moiety to ligation products), or otherwise reacting a terminal nucleotide (e.g., a ribose, a ribonucleotide, a reversibly terminated nucleotide, or a reversibly terminated ribonucleotide of a ligation product).

FIG. 11D illustrates formation of non-limiting reversible bonds by employing reversibly terminated nucleotides. Such nucleotides can include ribonucleotide-based forms having a 2′—OH group. In some embodiments, the method can include: introducing a reversibly terminated nucleotide to a capture probe or a target sequence (e.g., a ligation product), cleaving the reversibly terminated nucleotide to unblock a terminal nucleotide (e.g., by using a stimulus, such as light, pH, etc.), and optionally treating the terminal nucleotide with an oxidizing agent (e.g., sodium meta-periodate) to provide a capture moiety (e.g., including one or more aldehyde groups).

In some embodiments, the capture chemistry captures the ligation product but provides a junction that cannot be replicated by a polymerase. Non-limiting junctions include a click signature linkage (e.g., SPAAC [azide moiety and cyclooctyne moiety] or Diels-Alder [diene+dienophile, such as maleimide and furan], which may not be read-through by a polymerase), a streptavidin/biotin linkage, an avidin/biotin linkage, a maltose/maltose-binding protein linkage, a carbohydrate/carbohydrate-binding protein linkage, or an antigen/antibody linkage. Any capture moiety, reactive moiety, or capture chemistry described herein for a ligation product can be also used for other sequence (e.g., RTL probes).

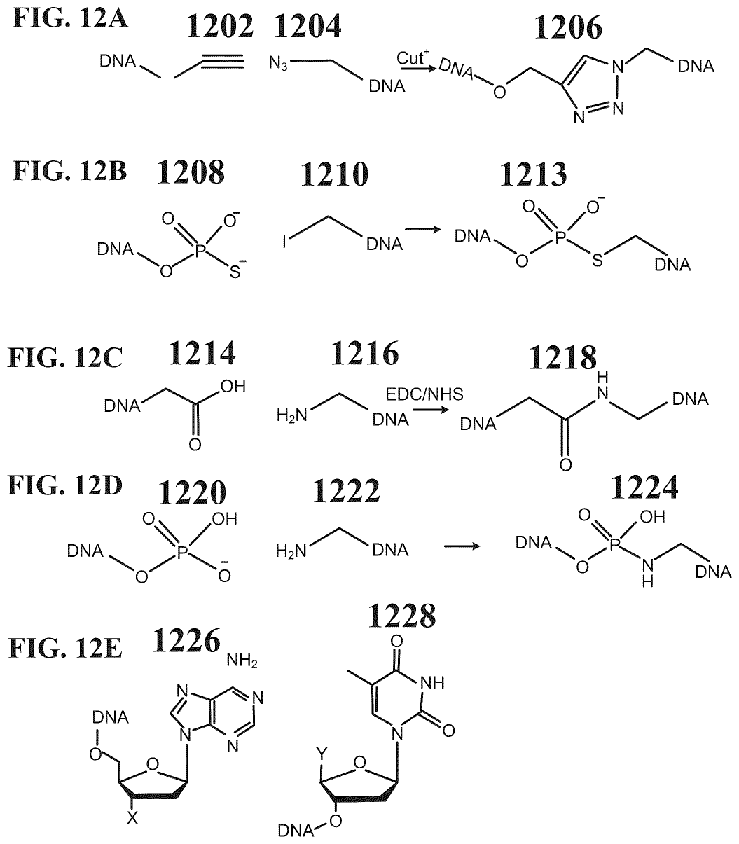

FIGS. 12A-12E exemplifies further representative reactions. FIG. 12A shows a chemical ligation reaction of an alkyne moiety 1202 and an azide moiety 1204 reacting under copper-mediated cycloaddition to form a triazole linkage 1206. FIG. 12B shows a chemical ligation reaction of a phosphorothioate group 1208 with an iodide group 1210 to form a phosphorothioate linkage 1212. FIG. 12C shows a chemical ligation reaction of an acid 1214 and amine 1216 to form an amide linkage 1218. FIG. 12D shows a chemical ligation reaction of a phosphate moiety 1220 and an amine moiety 1222 to form a phosphoramidate linkage 1224. FIG. 12E shows a conjugation reaction of two species 1226 and 1228.

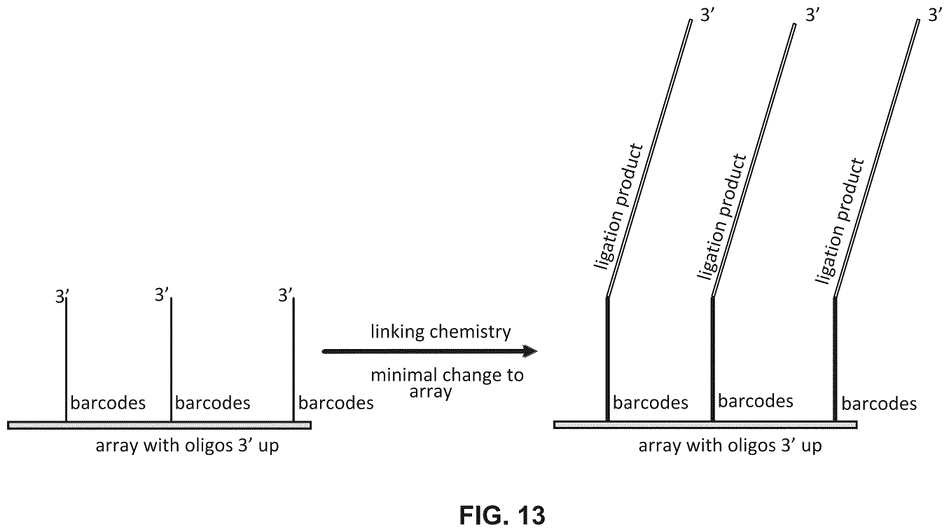

For instance, as seen in FIG. 13, the non-limiting workflow includes (a) providing a biological sample on an array comprising a plurality of barcoded capture probes and (b) covalently reacting the capture moiety of the capture probe with a reactive moiety on a ligation product to form a captured ligation product. Capture chemistry can be used to provide a junction between the capture probe and the ligation product, and further methods can include those that do not require replication by the polymerase through that junction. By forming a junction, a more stable capture probe/ligation product complex can be generated, which mitigates unwanted release of the capture ligation product and its subsequent mislocalization on the spatial array. In some embodiments, the non-limiting workflow can also allow for minimal changes to the array having the barcoded capture probes.

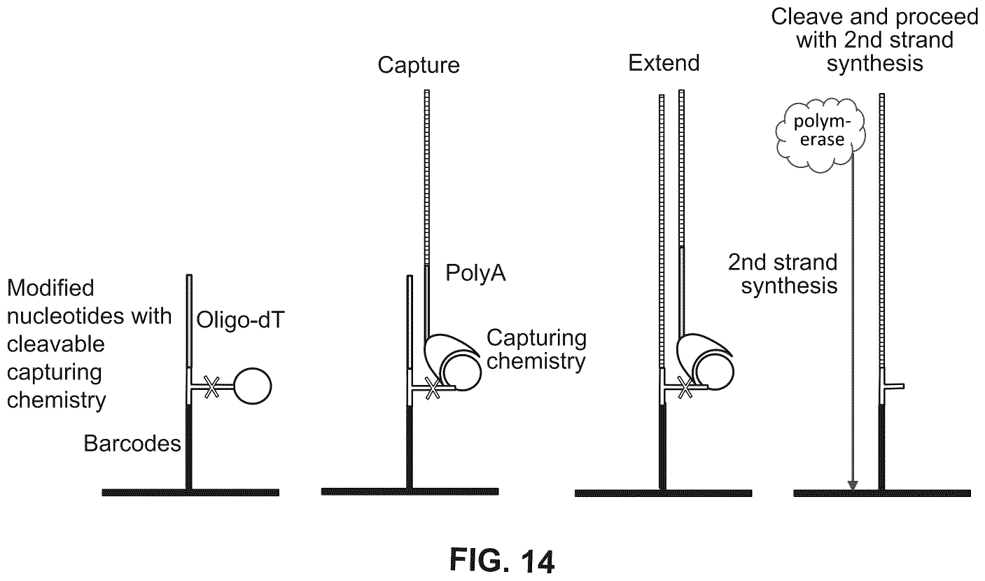

FIG. 14 shows a non-limiting workflow to provide a junction that is not configured for replication by a polymerase, as well as to provide a further product that can undergo further downstream analysis (e.g., second strand synthesis, sequencing, etc.). Such a further product can be the resultant product once the cleavage reaction has been conducted to remove the capture moiety and the ligation product.

As seen in FIG. 14, the capture probe can be configured to have a cleavable moiety that can be used to release the junction from the captured ligation product. In some embodiments, the capture probe can include: (i) a spatial barcode, (ii) a capture moiety, and (iii) a cleavable moiety in proximity to the capture moiety, wherein the cleavable moiety is configured to be cleaved after forming the junction. By forming a junction between the ligation product and the capture probe, a more stable capture probe/ligation product complex can be generated, which mitigates unwanted release of the capture ligation product and its subsequent mislocalization on the spatial array.

The capture probe can include a capture domain having various functional features. For example, and without limitation, the capture domain can include an oligo-dT sequence complementary to the poly(A) domain of the ligation product, as well as a capture moiety for conducting capture chemistry with a reactive moiety of the ligation product. The oligo-dT sequence can be used to prime extension of the capture probe using the captured ligation product as a template for the extension. In some embodiments, oligo-dT sequence can be used to further stabilize the binding of the ligation probe to the barcoded capture probe, when used in conjunction with the capturing chemistry.

In turn, the method can include: (a) providing a biological sample on an array comprising a plurality of capture probes; (b) covalently reacting the capture moiety with a reactive moiety of a ligation product to form a captured ligation product; (c) generating an extended ligation product by extending the capture probe; and (d) cleaving the cleavable moiety, thereby releasing the captured ligation product and the reaction moiety complex and junction.

The cleavable moiety can include photocleavable moieties, such as nitrobenzyl (NB), 6-nitropiperonyl (NP), anthryl-9-methyl (An), coumarin (CM), 7-(diethylamino) coumarin (DEACM), or 6-nitropiperonyloxymethyl (NPOM) groups, which can be removed by using ultraviolet (UV) light (e.g., at about 365 nm). Such photocleavable moieties can be provided to terminal nucleotide(s) of the capture probe, in which the photocleavable moiety is provided in proximity to a capture moiety. After reacting the capture moiety with a reactive moiety of the ligation product, the photocleavable moiety can be cleaved in the presence of UV light, thereby releasing the previously captured ligation product.

The extended ligation product can be generated in any useful manner. In one instance, the capture probe further includes a priming region. In use, the priming region can be extended by the polymerase using the sequence of the captured ligation product as the template. The extended ligation product now includes relevant information about the target analyte, and the ligation product can be released.

Upon cleaving the cleavable moiety, the captured ligation product is released, thereby providing an extended ligation product (or extension product) lacking the junction. As can be seen, the extended ligation product can include sequences that are complementary to sequences of the ligation product, as well as barcode sequences and other sequences as present on the capture probe. The method can include further downstream analysis of the extended ligation product (e.g., by way of second strand synthesis, sequencing, and the like).

Any of the methods described herein can further include generating an extended ligation product by binding to a priming region and extending the capture probe using the ligation product as a template, wherein the extended ligation product comprises complementary sequences of the ligation product; and cleaving the cleavable moiety, thereby releasing the captured ligation product and providing an extended ligation product lacking the junction, wherein the extended ligation product comprises complementary sequences of the ligation product in combination with the capture probe sequences. The operations of generating an extended ligation product and cleaving the cleavable moiety can occur after the operation of covalently reacting the capture moiety with a reactive moiety of a ligation product to form a captured ligation product.

In some embodiments, a chemical group (e.g., a cleavable moiety) could be added to the terminal nucleotide on the barcoded capture probe of array. One or more terminal nucleotides can be provided. The cleavable moiety can be cleaved off after the extension step, thereby allowing the polymerase to go through junction for second strand synthesis.

FIG. 15 provides another non-limiting workflow for providing a captured ligation product, extended ligation product, and further product. The first capture probe can include a capture domain having an oligo-dT sequence for hybridizing to the poly(A) domain of the ligation product. The second capture probe, which is located in proximity to the first capture probe, can include a capture moiety for conducting capture chemistry with a reactive moiety of the ligation product. The oligo-dT capture domain sequence of the first capture probe can hybridize to the ligation product and the second capture probe capture moiety can capture the ligation product reactive moiety due to their proximity, thereby generating a more stable capture probe/ligation product complex, which mitigates unwanted release of the captured ligation product and its potential mislocalization on the spatial array. The oligo-dT capture domain can further prime extension of the capture probe, thereby generating an extension product comprising the complement of the captured ligation product attached to the barcoded capture probe. For further processing, the cleavable moiety can be cleaved to release the captured ligation product thereby allowing for downstream processing, such as second strand synthesis of the extension product.

As seen in this non-limiting workflow of FIG. 15, the capturing chemistry can be used to capture strongly the RTL probes, and the oligo-dT sequence can be used for priming the extension step. In some embodiments, the oligo-dT sequence can be used to stabilize the binding of the RTL probe to the barcoded capture probe.

By having two (or more) different populations of oligonucleotides (e.g., barcoded capture probes) on the array features, chemistry could be simplified, as this configuration could permit capture on the 3′ end rather than a chemical moiety that is inserted midway up the oligonucleotide on a base. This configuration could encompass any number of chemical options for design.

Also described herein are methods for capturing a ligation product to mitigate mislocalization of a ligation product on a spatial array. Such methods can include: (a) providing a biological sample on a first substrate and an array comprising a plurality of capture probes, wherein a capture probe of the plurality of capture probes comprises: (i) a spatial barcode and (ii) a capture moiety, or wherein a first capture probe of the plurality of capture probes comprises (i) a spatial barcode and wherein a second capture probe of the plurality of capture probes comprises (ii) a capture moiety, and wherein the capture probe or the first capture probe is affixed to the first substrate or affixed to a second substrate; (b) hybridizing a first probe and a second probe to the analyte in the biological sample, wherein the first probe and the second probe each comprise one or more sequences that are substantially complementary to sequences of the analyte, and wherein the second probe comprises a reactive moiety; (c) generating a ligation product by ligating the first probe and the second probe, wherein the ligation product comprises the reactive moiety; (d) releasing the ligation product from the analyte; and (c) covalently reacting the capture moiety with the reactive moiety of the ligation product to form a captured ligation product on the substrate. In some embodiments, the capture probe or the first capture probe is affixed to the second substrate. In further embodiments, the method can include: (d) releasing the ligation product from the analyte, thereby transferring the ligation product from the first substrate to be captured by the second substrate.

In other embodiments, the method can further include, prior to step (d): aligning the first substrate with the second substrate comprising the array, such that at least a portion of the biological sample is aligned with at least a portion of the array. In some embodiments, the aligning includes: mounting the first substrate on a first member of a support device, the first member configured to retain the first substrate; mounting the second substrate on a second member of the support device; applying a reagent medium (e.g., any described herein) to the first substrate and/or the second substrate; and operating an alignment mechanism of the support device to move the first member and/or the second member such that at least a portion of the biological sample is aligned with at least a portion of the array, and such that the portion of the biological sample and the portion of the array contact the reagent medium. In some embodiments, the step (d) can further include: releasing the ligation product when at least a portion of the biological sample is aligned with at least a portion of the array. In other embodiments, the releasing step (d) includes: contacting the biological sample with a reagent medium comprising a permeabilization agent (e.g., any described herein, such as a protease) and an agent for releasing the ligation product (e.g., any described herein, such as a nuclease), thereby permeabilizing the biological sample and releasing the ligation product from the analyte.

Any of the methods described herein can further include, for example, as step (f), determining (i) all or a part of the sequence of the captured ligation product, or a complement thereof, and (ii) the sequence of the spatial barcode, or a complement thereof, and using the determined sequence of (i) and (ii) to identify a location of the analyte in the biological sample.

Also described herein are methods for enhancing the specificity of binding of a capture probe to a target sequence in a biological sample. In some embodiments, the method includes: (a) contacting the biological sample with a substrate, wherein the substrate includes a plurality of capture probes affixed to the substrate, and wherein the capture probe includes a spatial barcode and a capture domain (e.g., or a capture moiety); (b) binding a first probe and a second probe to a target analyte in the biological sample, wherein: the first probe and the second probe are substantially complementary to adjacent sequences of the target analyte, the second probe includes a capture probe binding domain (e.g., or a reactive moiety) that binds to or react with the capture domain (e.g., or the capture moiety) of the capture probe, wherein the capture probe binding domain is optionally blocked and prevented from hybridizing to the capture domain (e.g., or the capture moiety) of the capture probe affixed to the substrate; (c) ligating the first probe and the second probe, thereby creating a ligation product that is substantially complementary to the target analyte; (d) releasing: the ligation product from the analyte, and the block from the capture probe binding domain, thereby allowing the capture probe binding domain to bind to the capture domain of the capture probe on the substrate, thereby enhancing the specificity of binding of a polynucleotide to a target analyte in a biological sample.

Also provided herein are methods for determining (i) all or a part of the sequence of the ligated probe specifically bound to or specifically reacted with the capture domain (e.g., or the capture moiety), or a complement thereof, and (ii) the sequence of the spatial barcode, or a complement thereof, and using the determined sequence of (i) and (ii) to identify a location of a target analyte in the biological sample.

Any of the methods described herein can further include contacting a biological sample with a substrate, wherein the capture probe is affixed to the substrate (e.g., immobilized to the substrate, directly or indirectly). In some embodiments, the capture probe includes a spatial barcode and the capture domain. In some embodiments, prior to hybridization, a block is released from the capture binding domain of the ligated probes. The ligated probe then binds to the capture domain of the capture probe. After hybridization of the ligated probe to the capture probe, the ligated probe is analyzed (e.g., the sequence is determined) using methods disclosed herein, including but not limited to extension of the probe, RT, addition of adaptors, and sequencing.

Further exemplary methods and compositions relating to RTL, which can be used to produce ligation products for capture, are described in PCT Publication No. WO2021/133849 and/or U.S. Patent Application Publication No. 2022/0282329 A1 and/or U.S. Patent Application No. 62/952,736, each of which is incorporated herein by reference in their entireties.

### Junctions

In some embodiments, the junction is configured for replication by a polymerase. Non-limiting junctions can include a click signature linkage, a triazole linkage, an isoxazoline linkage, an S-phosphorothioester linkage, a phosphorothioate linkage, an amide linkage, a disulfide linkage, a boranophosphate linkage, a phosphoramidate linkage, a urea linkage, or a squaramide linkage.

In some embodiments, the junction is not configured for replication by a polymerase. Non-limiting junctions can include a click signature linkage (e.g., SPAAC [azide moiety and cyclooctyne moiety] or Diels-Alder [diene+dienophile, such as maleimide and furan], which may not be read-through by a polymerase), a streptavidin/biotin linkage, an avidin/biotin linkage, a maltose/maltose-binding protein linkage, a carbohydrate/carbohydrate-binding protein linkage, or an antigen/antibody linkage.

In some embodiments, a functional oligonucleotide (e.g., to introduce a common feature, reactive group, or moiety to capture probes) is ligated to capture probes and/or ligation products. In use, the functional oligonucleotide can provide any useful capture chemistry, capture moieties, or reactive moieties to provide one or more junctions.

The junction can include one or more chemical signatures. In one embodiment, the chemical signature includes a click-chemistry signature, which arises from reacting a click-chemistry reaction pair (e.g., any described herein). Non-limiting examples of click-chemistry signatures include a triazole, an unsaturated six-member ring, a covalent bond, and the like.

In another embodiment, the chemical signature can include a reaction signature, which arises from reacting a cross-linker reaction pair. Non-limiting examples of cross-linker reaction pairs include those for forming a covalent bond between a carboxyl group (e.g., —CO₂H) and an amino group (e.g., —NH₂); or between an imido group (e.g., malcimido or succinimido) and a thiol group (e.g., —SH); or between an epoxide group and a thiol group (e.g., —SH); or between an epoxide group and an amino group (e.g., —NH₂); or between an ester group (e.g.,
—CO₂R, in which R is an organic moiety, such as optionally substituted alkyl, aryl, etc.) and an amino group (e.g., —NH₂); or between an carbamido group (e.g., —NHC(O) Het, where Het is a N-containing heterocyclyl) and an amino group (e.g., —NH₂); or between a phospho group (e.g.,
—P(O)(OH)₂) and an amino group (e.g., —NH₂), such as 1-ethyl-3-(3-dimethylaminopropyl) carbodiimide (EDC) and dicyclohexylcarbodiimide (DCC), optionally used with N-hydroxysuccinimide (NHS) and/or N-hydroxysulfosuccinimide (sulfo-NHS); or between a thiol group (e.g., —SH) and an alkenyl group. Other cross-linkers include those for forming a covalent bond between an amino group (e.g., —NH₂) and a thymine moiety, such as succinimidyl-[4-(psoralen-8-yloxy)]-butyrate (SPB); a hydroxyl group (e.g., —OH) and a sulfur-containing group (e.g., free thiol, —SH, sulfhydryl, cysteine moiety, or mercapto group), such as p-maleimidophenyl isocyanate (PMPI); between an amino group (e.g., —NH₂) and a sulfur-containing group (e.g., free thiol, —SH, sulfhydryl, cysteine moiety, or mercapto group), such as succinimidyl 4-(p-maleimidophenyl) butyrate (SMPB) and/or succinimidyl 4-(N-maleimidomethyl)cyclohexane-1-carboxylate (SMCC); between a sulfur-containing group (e.g., free thiol, —SH, sulfhydryl, cysteine moiety, or mercapto group) and a carbonyl group (e.g., an aldehyde group, such as for an oxidized glycoprotein carbohydrate), such as N-beta-maleimidopropionic acid hydrazide-trifluoroacetic acid salt (BMPH), 3-(2-pyridyldithio) propionyl hydrazide (PDPH), and/or a 3-(2-pyridyldithio) propionyl group (PDP); and between a maleimide-containing group and a sulfur-containing group (e.g., free thiol, —SH, sulfhydryl, cysteine moiety, or mercapto group). Yet other cross-linkers include those for forming a covalent bond between two or more unsaturated hydrocarbon bonds, e.g., mediated by radical polymerization, such as a reaction of forming a covalent bond between a first alkene group and a second alkene group (e.g., a reaction between acrylate-derived monomers to form a polyacrylate, polyacrylamide, etc.); those for forming a phosphoramidate bond with a phosphorous-containing group (e.g., a phosphate group) and a nitrogen-containing group (e.g., an amino group); or those for conducting a nucleophilic substitution reaction. Each functional group within a cross-linker reaction pair can be used as a capture moiety or a reactive moiety. For instance, the capture moiety and the reactive moiety, taken together, can each be a different member of a cross-linker reaction pair.

The junction can be formed between reaction pairs. In one embodiment, the reaction pair is one of a click-chemistry reaction pair, which can include a first click-chemistry group and a second click-chemistry group that reacts with that first click-chemistry group. Exemplary click-chemistry groups include, e.g., a click-chemistry group, e.g., one of a click-chemistry reaction pair selected from the group consisting of a Huisgen 1,3-dipolar cycloaddition reaction between an alkynyl group and an azido group to form a triazole-containing linker; a cycloaddition reaction such as a strain-promoted azide-alkyne cycloaddition, a copper-catalyzed azide-alkyne cycloaddition, a strain-promoted alkyne-nitrone cycloaddition, a Diels-Alder reaction, a [3+2] cycloaddition, a [4+2] cycloaddition, or a [4+1] cycloaddition; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; a splint ligation reaction with a phosphorothioate group and an iodo group; a thiol-ene reaction between a thiol group and an alkenyl group; and a reductive amination reaction with an aldehyde group and an amino group. In some embodiments, the capture moiety and the reactive moiety, taken together, can each be a different member of a reaction pair.

A junction can be formed between reactive groups. Exemplary reactive groups include an amino (e.g., —NH₂), a thio (e.g., a thioalkoxy group or a thiol group), a hydroxyl, an ester (e.g., an acrylate), an imido (e.g., a maleimido or a succinimido), an epoxide, an isocyanate, an isothiocyanate, an anhydride, an amido, a carbamido (e.g., a urea derivative), an azide, an optionally substituted alkynyl, or an optionally substituted alkenyl. Yet other reactive groups include azides, alkynes, nitrones (e.g., 1,3-nitrones), strained alkenes (e.g., trans-cycloalkenes such as cyclooctenes or oxanorbornadiene), tetrazines, tetrazoles, iodides, thioates (e.g., phosphorothioate), acids, amines, and phosphates. Each of the capture moiety and the reactive moiety can be, independently, any reactive group described herein.

In other embodiments, the junction can include a binding reaction signature, which arises from reacting a binding reaction pair. Exemplary binding groups and binding reaction pairs include those for forming a covalent bond between biotin and avidin, biotin and streptavidin, biotin and neutravidin, desthiobiotin and avidin (or a derivative thereof, such as streptavidin or neutravidin), hapten and an antibody, an antigen and an antibody, a primary antibody and a secondary antibody, and lectin and a glycoprotein.

### Capture Moieties

A capture probe can have any useful capture moiety. Non-limiting capture moieties include an azide moiety, an alkyne moiety, a phosphorothioate moiety, a leaving moiety (e.g., an iodide), a carboxyl moiety (e.g., —CO₂H), an amino moiety (e.g., —NR₁R₂), a phosphate moiety, a thiol moiety, streptavidin, avidin, or biotin. Other capture moieties are described herein (e.g., members of a cross-linker reaction pair, members of a reaction pair, or reactive groups).

Capture moieties can be attached to capture probes by any useful manner. The capture moiety can be attached to the capture probe either before or after the capture probe is attached to the array. For instance, capture probes can be synthesized or prepared to include capture moieties, and then the capture probes are immobilized on a surface of the arrayed substrate.

Alternatively, methods can be optimized for use with arrays having existing capture probes. For example, methods for converting or modifying the capture probe (e.g., a terminal nucleotide of a capture probe) can be used to introduce a capture moiety. Any of the methods described herein can further include converting a terminal nucleotide of the capture probe to become the capture moiety. Converting a terminal nucleotide to become a capture moiety can include treating the terminal nucleotide with an oxidizing agent (e.g., sodium meta-periodate) thereby generating a capture moiety comprising one or more aldehyde groups.

In some embodiments, capture probes can be modified to enzymatically add biotin-dUTP to the end of probes in an array. Non-limiting enzymes can include using terminal deoxynucleotidyl transferase (TdT).

Capture probes can be provided in any useful manner, such as having its 3′ end distal to a surface of the array (e.g., a “3′ up” array) or having its 5′ end distal to a surface of the array (e.g., a “5′ up” array). Any useful combination of capture moieties (for capture probes) and reactive moieties (for ligation products) can be employed to provide a captured ligation product. Chemistries described herein for 3′ up arrays can be adapted for use with 5′ up arrays.

In some embodiments, a functional oligonucleotide (e.g., to introduce a common feature, reactive group, or moiety to capture probes) is ligated to capture probes and/or ligation products. In some embodiments, the method can include: providing an array comprising a plurality of capture probes; and ligating a functional oligonucleotide to a 3′ or 5′ end of the capture probe to provide a capture moiety.

In some embodiments, a capture probe can include a cleavage domain (e.g., a cleavable moiety) and/or a functional domain (e.g., a primer-binding site, unique molecular identifier, sequencing domains, etc.). A cleavable moiety can be positioned, for example, between the barcode and the substrate (e.g., an array), between the capture moiety and the substrate (e.g., an array), or between the capture moiety and the barcode. In some instances, the cleavable moiety can facilitate isolation of a product (e.g., an extended ligation product) to be used in downstream applications. In some embodiments, the cleavable moiety can be in proximity to a surface of the substrate (e.g., an array) or feature. In some embodiments, the cleavable moiety can be distal to a surface of the array. In some embodiments, the capture moiety is positioned at the 3′ end of the capture probe, and the reactive moiety is positioned on the 5′ end of the captured sequence or captured intermediate agent (e.g., the ligation product). In some embodiments, the capture moiety is positioned at the 5′ end of the capture probe, and the reactive moiety is positioned on the 3′ end of the captured sequence or captured intermediate agent (e.g., the ligation product). In other embodiments, the capture moiety is positioned at the 3′ end of the capture probe, and the reactive moiety is positioned on the 3′ end of the captured sequence or captured intermediate agent (e.g., the ligation product). In some embodiments, the cleavable moiety is disposed between a surface of the array and the spatial barcode.

In any of the embodiments, a cleavable moiety can include a cleavage domain (e.g., disulfide linker, sequence recognized and cleaved by a uracil-DNA glycosylase, an apurinic/apyrimidinic (AP) endonuclease (APEI), a uracil-specific excision reagent (USER), an endonuclease VIII, etc.).

In some embodiments, the capture probe can include a priming region (e.g., a priming region configured to be provided in proximity to the junction or a priming region on the 3′ end of the capture probe). In some embodiments, the priming region is downstream of the capture moiety. In some embodiments, the capture probe can include a priming region and a cleavable moiety. In some embodiments, the priming region and the cleavable moiety are disposed in proximity to the priming region. See, e.g., Section (II) (b) (e.g., subsections (i)-(vi)) of PCT Publication No. WO2020/176788 and/or U.S. Patent Application Publication No. 2020/0277663. Generation of capture probes can be achieved by any appropriate method, including those described in Section (II) (d) (ii) of PCT Publication No. WO2020/176788 and/or U.S. Patent Application Publication No. 2020/0277663.

In some embodiments, the capture probe includes a cleavable moiety in proximity to the capture moiety, and the cleavable moiety is configured to be cleaved after forming the junction (e.g., the junction with the reactive moiety). In some embodiments, the capture probe (e.g., a second capture probe) can include a cleavable moiety in proximity to a surface of a substrate, such as an array. In some embodiments, the capture probe can include a cleavable moiety in proximity to the capture moiety. In some embodiments, the cleavable moiety is configured to be cleaved after forming the junction (e.g., the junction with the reactive moiety).

The cleavable moiety can be provided within a linker. In some embodiments, the linker can include one or more transcribable elements, such as a nucleotide or a nucleic acid. In other embodiments, the linker can include a chemical linker. Linkers can include a bond (e.g., a covalent bond); an optionally substituted atom (e.g., methylene (such as, e.g., —CR₂—, in which each R is, independently, H or C₁₋₁₂ alkyl), oxy [—O—], thio [—S—], imino (e.g., —NR—, in which R is H or C₁₋₁₂ alkyl), carbonyl [—C(O)—], amido (e.g., —C(O) NR— or —NRC(O)—, in which R is H or C₁₋₁₂ alkyl), sulfinyl [—SO—], sulfonyl [—SO₂—], and the like); an amino acid; a plurality of amino acids; a nucleotide; a plurality of nucleotides; an optionally substituted alkylene (e.g., an optionally substituted C₁₋₂₅ alkylene); and optionally substituted heteroalkylene (e.g., poly(ethylene glycol), such as —(OCH₂CH₂)ₙ—, in which n is an integer of 1 to 100); an optionally substituted arylene (e.g., phenylene, phenanthrylene, phthaloyl, naphthylene, and the like); or an optionally substituted heteroarylene, as well as combinations thereof.

### Reactive Moieties

Described herein are methods for capturing an intermediate agent such as a ligation product using reactive moieties that interact with capture moieties. Any of the methods described herein can include providing a biological sample on an array comprising a plurality of any of the capture probes described herein and covalently reacting the capture probe with a reactive moiety of an intermediate agent, such as a ligation product, to form a captured intermediate agent, such as a captured ligation product. In some cases, the ligation product comprises a sequence that is substantially complementary and can serve as a proxy for an analyte in the biological sample.

A reactive moiety can be attached to a target sequence or an intermediate agent (e.g., a ligation product). When a capture moiety and a reactive moiety react, a junction is formed that connects the target sequence or the intermediate agent to the capture probe. The junction can include a variety of chemistries that combine two different agents (e.g., the capture probe and the target sequence or intermediate agent). For example, a reactive moiety can include an azide moiety, an alkyne moiety, a phosphorothioate moiety, a leaving moiety (e.g., an iodide), a carboxyl moiety (e.g., —CO₂H), an amino moiety (e.g., —NR₁R₂), a phosphate moiety, a thiol moiety, streptavidin, avidin, or biotin.

Any of the methods described herein can further include converting a terminal nucleotide of the ligation product to provide the reactive moiety. Converting a terminal nucleotide to provide a reactive moiety can include treating the terminal nucleotide with an oxidizing agent (e.g., sodium meta-periodate) to provide the reactive moiety comprising one or more aldehyde groups.

Ligation products can be generated in any useful manner. Any of the methods described herein can further include hybridizing the first probe and the second probe to the target analyte; generating the ligation product by ligating the first probe and the second probe; and releasing the ligation product from the target analyte.

In some embodiments, RTL probes (e.g., the first probe and/or the second probe) may each comprise a reactive moiety such that, upon hybridization to the target and exposure to appropriate ligation conditions, the RTL probes may ligate to one another. In some embodiments, an RTL probe that includes a reactive moiety is ligated chemically. For example, a probe capable of hybridizing to a sequence 3′ of a target sequence (e.g., a first target region) of a nucleic acid molecule may comprise a first reactive moiety, and a probe capable of hybridizing to a sequence 5′ of a target sequence (e.g., a second target region) of the nucleic acid molecule may comprise a second reactive moiety. When the first and second probes are hybridized to the first and second target regions of the nucleic acid molecule, the first and second reactive moieties may be adjacent to one another.

In other embodiments, one or more of the RTL probes (e.g., the first probe and the second probe) may comprise a reactive moiety such that, in the presence of the capture probe and exposure to appropriate reaction conditions, the probe(s) and the capture probe may react with one another.

A reactive moiety on an RTL probe may be selected from the non-limiting group consisting of azides, alkynes, nitrones (e.g., 1,3-nitrones), strained alkenes (e.g., trans-cycloalkenes such as cyclooctenes or oxanorbornadiene), tetrazines, tetrazoles, iodides, thioates (e.g., phosphorothioate), acids, amines, and phosphates. For example, the first reactive moiety of a first RTL probe may comprise an azide moiety, and a second reactive moiety of a second RTL probe may comprise an alkyne moiety. The first and second reactive moieties may react to form a linking moiety. A reaction between the first and second reactive moieties may be, for example, a cycloaddition reaction such as a strain-promoted azide-alkyne cycloaddition, a copper-catalyzed azide-alkyne cycloaddition, a strain-promoted alkyne-nitrone cycloaddition, a Diels-Alder reaction, a [3+2] cycloaddition, a [4+2] cycloaddition, or a [4+1] cycloaddition; a thiol-ene reaction; a nucleophilic substitution reaction; or another reaction. In some cases, reaction between the first and second reactive moieties may yield a triazole moiety or an isoxazoline moiety. A reaction between the first and second reactive moieties may involve subjecting the reactive moieties to suitable conditions such as a suitable temperature, pH, or pressure and providing one or more reagents or catalysts for the reaction. For example, a reaction between the first and second reactive moieties may be catalyzed by a copper catalyst, a ruthenium catalyst, or a strained species such as a difluorooctyne, dibenzylcyclooctyne, or biarylazacyclooctynone. Reaction between a first reactive moiety of a first probe hybridized to a first target region of the nucleic acid molecule and a second reactive moiety of a third probe hybridized to a second target region of the nucleic acid molecule may link the first probe and the second probe to provide a ligated probe (or ligation product). Upon linking, the first and second RTL probes may be considered ligated. Accordingly, reaction of the first and second reactive moieties may comprise a chemical ligation reaction such as a copper-catalyzed 5′ azide to 3′ alkyne “click” chemistry reaction to form a triazole linkage between two probes. In other non-limiting examples, an iodide moiety may be chemically ligated to a phosphorothioate moiety to form a phosphorothioate bond, an acid may be ligated to an amine to form an amide bond, and/or a phosphate and amine may be ligated to form a phosphoramidate bond. Any reactive moieties described herein for probes may be used as a capture moiety for a capture probe, and any probe having such any reactive moieties can be reacted with a capture moiety of a capture probe.

In some embodiments, after ligation of the first and second probes to create the ligation product, the ligation product is released from the analyte. In some embodiments, the ligation product is released enzymatically. In some embodiments, an endoribonuclease is used to release the ligation product from the analyte. In some embodiments, the endoribonuclease is one or more of RNase H, RNase A, RNase C, or RNase I.

In some instances, the endoribonuclease is RNase H. RNase H is an endoribonuclease that specifically hydrolyzes the phosphodiester bonds of RNA, when hybridized to DNA. The RNases H are a conserved family of ribonucleases which are present many different organisms. There are two primary classes of RNase H: RNase H1 and RNase H2. Retroviral RNase H enzymes are similar to the prokaryotic RNase H1. All of these enzymes share the characteristic that they are able to cleave the RNA component of an RNA: DNA heteroduplex. In some embodiments, the RNase H is RNase H1, RNase H2, or RNase H1, or RNase H2. In some embodiments, the RNase H includes but is not limited to RNase HII from Pyrococcus furiosus, RNase HII from Pyrococcus horikoshi, RNase HI from Thermococcus litoralis, RNase HI from Thermus thermophilus, RNase HI from E. coli, or RNase HII from E. coli.

In some embodiments, after generation of a ligation product, the biological sample is permeabilized. In some embodiments, permeabilization occurs using a protease. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin.

### Kits

The present disclosure also features kits to capture target analytes with the methods described herein. Thus, provided herein are kits including a substrate including a plurality of capture probes, where a capture probe of the plurality of capture probes includes: (i) a spatial barcode and (ii) a capture moiety; a plurality of templated ligation probes including a first probe and a second probe, where a templated ligation probe of the plurality of templated ligation probes includes a reactive moiety; where the capture moiety is configured to covalently react with the reactive moiety to form a junction configured for replication by a polymerase, and where the templated ligation probe includes a sequence that is complementary to a sequence of an analyte in a biological sample.

Also provided herein are kits including: a substrate including a plurality of capture probes, where a capture probe of the plurality of capture probes includes: (i) a spatial barcode and (ii) a capture moiety (e.g., any of the capture moieties described herein), or where a first capture probe of the plurality of capture probes includes: (i) a spatial barcode and where a second capture probe of the plurality of capture probes includes (ii) a capture moiety (e.g., any of the capture moieties described herein); a plurality of templated ligation probes comprising a first probe and second probe, where a templated ligation probe of the plurality of templated ligation probes includes a reactive moiety (e.g., any of the reactive moieties described herein); where the capture moiety is configured to covalently react with the reactive moiety to form a junction that is not configured for replication by a polymerase, and where the templated ligation probe includes a sequence that is complementary to a sequence of an analyte in a biological sample.

In some embodiments, the kit includes instructions for performing any of the methods described herein.

In some embodiments, the capture probe, the first capture probe, and/or the second capture probe, if present, includes a cleavable moiety.

In some embodiments, each of a pair of templated ligation probes includes a sequence that is complementary to adjacent or non-adjacent sequences of an analyte.

What is claimed is:

1. A method for capturing a ligation product, the method comprising:
(a) providing an array comprising a plurality of capture probes, wherein a capture probe of the plurality of capture probes comprises: (i) a spatial barcode and (ii) a capture moiety; and
(b) covalently reacting the capture moiety of the capture probe with a reactive moiety of the ligation product to form a captured ligation product, wherein the ligation product is generated by ligating a first probe and a second probe, wherein the first probe and the second probe each comprise one or more sequences that are substantially complementary to sequences of a nucleic acid in a biological sample, and
wherein the capture moiety and the reactive moiety react together to form a junction configured for replication by a polymerase.

2. The method of claim 1, wherein the one or more sequences are adjacent to one another.

3. The method of claim 1, wherein there is a gap between the one or more sequences, and wherein the first probe or the second probe is extended until the first probe and the second probe abut prior to ligating.

4. The method of claim 1, further comprising releasing the ligation product from the nucleic acid.

5. The method of claim 1, wherein the junction comprises a click signature linkage, a triazole linkage, an isoxazoline linkage, an S-phosphorothioester linkage, a phosphorothioate linkage, an amide linkage, a disulfide linkage, a boranophosphate linkage, a phosphoramidate linkage, a urea linkage, or a squaramide linkage.

6. The method of claim 1, further comprising, before (a): converting a terminal nucleotide of the capture probe to generate the capture moiety.

7. The method of claim 6, wherein the converting comprises treating the terminal nucleotide with an oxidizing agent to generate the capture moiety comprising one or more aldehyde moieties.

8. The method of claim 7, wherein the terminal nucleotide comprises a ribose, a ribonucleotide, a reversibly terminated nucleotide, or a reversibly terminated ribonucleotide.

9. The method of claim 1, wherein the biological sample is disposed on the array.

10. The method of claim 1, wherein the biological sample is disposed on a substrate and further comprises aligning the substrate with the array, such that at least a portion of the biological sample is aligned with at least a portion of the array.

11. A method for capturing a ligation product, the method comprising:
(a) providing an array comprising a plurality of capture probes, wherein a capture probe of the plurality of capture probes comprises: (i) a spatial barcode and (ii) a capture moiety;
(b) covalently reacting the capture moiety with a reactive moiety of the ligation product to form a captured ligation product,
wherein the ligation product is generated by ligating a first probe and a second probe, wherein the first probe and the second probe each comprise one or more sequences that are substantially complementary to sequences of a nucleic acid in a biological sample, and
wherein the capture moiety and the reactive moiety react together to form a junction that is not configured for replication by a polymerase.

12. The method of claim 11, wherein the one or more sequences are adjacent to one another.

13. The method of claim 11, wherein there is a gap between the one or more sequences, and wherein the first probe or the second probe is extended until the first probe and the second probe abut prior to ligating.

14. The method of claim 11, further comprising releasing the ligation product from the nucleic acid.

15. The method of claim 11, wherein the junction comprises a click signature linkage, a streptavidin/biotin linkage, an avidin/biotin linkage, a maltose/maltose-binding protein linkage, a carbohydrate/carbohydrate-binding protein linkage, or an antigen/antibody linkage.

16. The method of claim 11, wherein the capture probe further comprises a cleavable moiety in proximity to the capture moiety, and wherein the cleavable moiety is configured to be cleaved after forming the junction.

17. The method of claim 16, wherein the capture probe further comprises a priming region located in proximity to the junction.

18. The method of claim 17, further comprising, after (b):
(c) generating an extended ligation product by binding to the priming region and extending the capture probe, wherein an extended capture probe comprises sequences that are complementary to sequences of the ligation product; and
(d) cleaving the cleavable moiety, thereby releasing the captured ligation product and providing a further product lacking the junction.

19. The method of claim 11, wherein the biological sample is disposed on the array.

20. The method of claim 11, wherein the biological sample is disposed on a substrate and further comprises aligning the substrate with the array, such that at least a portion of the biological sample is aligned with at least a portion of the array.
